(19) United States
(12) Patent Application Publication
(10) Pub. No.: US 2010/0150923 A1
(43) Pub. Date: Jun. 17, 2010

(54) FUSION PROTEINS OF RECOMBINANT SARS CORONAVIRUS STRUCTURAL PROTEINS, THEIR PRODUCTION AND USES

(75) Inventors: Chengyu Jiang, Beijing (CN); Feng Guo, Beijing (CN); Shuan Rao, Beijing (CN); Bing Guan, Beijing (CN); Yi Huan, Beijing (CN); Peng Yang, Beijing (CN)

Correspondence Address: Workman Nydegger;1000 Eagle Gate Tower, 60 East South Temple, Salt Lake City, UT, 84111, US

(73) Assignee: Chinese Academy of Medical Sciences, Institute of Basic Medical Sciences, Beijing (CN)

(21) Appl. No.: 11/993,176

(22) PCT Filed: Jun. 13, 2006
(86) PCT No.: PCT/CN06/01293

§ 371 Date: Aug. 20, 2008

(30) Foreign Application Priority Data
Jun. 20, 2005 (CN) 200510077258.2
Jul. 7, 2005 (CN) 200510082719.5

(51) Int. Cl. A61K 39/42; C07K 14/00; C07K 16/00; C07H 21/04; C12N 15/63; C12N 5/10; C12P 21/04; A61K 39/12

(52) U.S. Cl. 424/134.1; 530/350; 530/387.3; 536/23.72; 435/320.1; 435/325; 435/358; 435/363; 435/328; 435/69.7; 435/69.6; 424/192.1; 424/186.1

(57) ABSTRACT

Fusion proteins of recombinant SARS coronavirus structural proteins, their production and uses are provided. An optimized SARS coronavirus S protein gene which can be highly expressed in the mammalian cell strains and SARS coronavirus S protein variants comprising deletion, modification or mutation amino acids 318-510 corresponding to SARS coronavirus S protein are also provided.

### FIELD OF THE INVENTION

The present invention relates to the fusion protein of the structural proteins of SARS-CoV virus and large scale expression in mammalian cells, the use of the fusion protein for manufacturing gene engineered vaccines and medications for preventing and treating the infection of SARS-CoV virus, and a kit for application of detecting the SARS-CoV virus infection comprising the said fusion protein. Furthermore. The present invention also relates to the finding of the toxic fragment in the S protein, which is one kind of structural proteins of SARS-CoV virus, and to varieties of vaccines designed for prophylaxis the SARS-CoV virus infection.

### BACKGROUND OF THE INVENTION

The pathogen of Severe Acute Respiratory Syndrome (Severe Acute Respiratory Syndrome, SARS) is a new type of coronavirus (SARS-CoV), which is featured on its widespread hosts, the rapid speed of spreading, ability to pass by droplets even by air, is greatly harmful to human beings. Therefore, the research of prophylaxis, treatment and detecting the SARS-CoV virus infection is still pressing.

Human as an object of the SARS-CoV virus vaccine, the stability and safety of the vaccine is a basic and most important requirement. As the research of gene-engineered vaccine is comparatively mature, it meets the requirements best.

There are four kinds of structural proteins, S, M, N, E in SARS-CoVvirus. The results of the experimental bioinformatics indicate that S protein and N protein have strong immunogenicity, thus, which are the main antigens in the vaccine research. M protein and E protein also have certain extent of immunogenicity, both are capable of being effective vaccines. S protein has the greatest probability to produce effective vaccines.

Yet according to the characteristics of S protein itself, there are numerous difficulties in successfully expressing and purifying the full-length and active S protein.

Due to there are a great deal of modification sites in post-translated S protein, mainly the glycosylation sites, the proteins expressed in the prokaryotic cells or yeast cells can not be folded correctly, resulting in influencing the activity of the said proteins. Only expressed in the mammalian cells, will the S protein be modified, folded and processed in the proper way. The protein produced in this way will be similar to its natural state; otherwise, it will seriously impact the effect of the immunity. Yet the expression of S protein in mammalian expression system, which is coded by S antigen, is very low and hardly possible in practical application.

Therefore, in order to produce effective SARS-CoV virus vaccines, three solutions should be settled. Firstly, the S protein of SARS-CoV virus should be expressed effectively in mammalian cells, and proper conditions should be selected to allow the proteins to be separated efficiently from the proteins and DNA of the host cells. Secondly, the yield of S protein expression should be increased so as to make a more economical production of vaccine. Thirdly, the safety of the vaccine must be ensured. As the study of the pathogenesis of SARS-CoV is limited, it is not known that how SARS-CoV causes acute lung injury, function failure of heart, and immune system breakdown. The problem of security risk existing in the production of SARS vaccine to date is still not solved by prior art, so it also needs further safe and effective SARS-CoV virus vaccines.

### SUMMARY OF THE INVENTION

To overcome the deficiencies of the prior art, the chief object of the present invention is to provide a gene fragment of S protein of SARS-CoV virus that can be expressed in mammalian cell lines.

The second object of the present invention is to largely express the structural proteins and their fusion proteins in cleavage form in mammalian cells and purify the said proteins.

The third object of the present invention is to provide gene-engineered vaccines for prophylaxis SARS-CoV virus infection by using the fusion proteins of the structural proteins of SARS-CoV virus, including the fusion protein of S protein. The inventors find that the binding of S protein and its receptor ACE2 could cause or aggravate the Acute Respiratory Distress Syndrome, as a result, it needs to delete or modify the binding fragment of S protein combining with ACE2 for developing safe and effective vaccines.

The fourth object of the present invention is to provide a kit for detecting SARS-CoV virus infection comprising of the obtained fusion protein of structural proteins of SARS-CoV virus.

The fifth object of the present invention is the use of the obtained fusion protein of S protein for manufacturing or screening medicaments for treating the SARS-CoV virus infection.

The sixth object is to provide vaccines for prophylaxis of the SARS-CoV virus infection, including DNA vaccines, protein vaccines, and virus carrier vaccines etc, by removing, mutating S protein of SARS-CoV virus, or modifying the amino acid sequence of fragment from 318 to 510, to cause S protein unable to bind to ACE2.

In the invention, the cleavage form of S protein is expressed as term “Sa-b”, which means the amino acids of the protein beginning from a site to b site in the full-length sequence of S protein of wild-type SARS CoV virus.

When “a” is the first amino acid as beginning, it will be expressed as “Sb”.

For example, S318-510 means that the amino acid sequence of the expressed protein is the amino acids from site 318 to 510 of the full-length sequence of S protein, S511 means that the amino acid sequence of the expressed protein is the amino acids from site 1 to 511 of the full-length sequence of S protein, S685 means that the amino acid sequence of the expressed protein is the amino acids from site 1 to 685 of the full-length sequence of S protein, and the like.

The technical solutions of the present invention include:

A fusion protein of structural proteins of SARS-CoV virus having a formula of X-Y-Z, wherein,

X comprises structural protein S, M, E or N of SARS-CoV virus, or random cleavage forms of these structural proteins. The structural protein S of SARS-CoV virus comprises any amino acids fragment that any of the amino acid from 318 to 510 is removed, modified or mutated, or fragment that the amino acid from 318 to 510 is removed, modified or mutated.

Y is a linking part consisting of 0 to 20 of any amino acids.

Z is a Fc, its variants of human IgG₁ including hinge region, CH₂, and CH₃ region or protein tags.

The protein tag includes but not limits to the 6×His tag, PEG tag and HAS (Human serum albumin) tag.

The S structural protein of SARS-CoV virus includes S protein in full lengthy or any cleaved form thereof.

The S structural protein of SARS-CoV virus is protein unable to bind to its receptor ACE2 or weakened the binding ability.

Preferably, Y indicates two amino acids, the said amino acids are lysine and arginine.

The present invention also involves a gene encoding S protein of SARS-CoV virus which can be expressed in mammalian cell lines, it is characterized in that the sequence of the said gene is SEQ ID NO: 1.

The present invention further involves a recombined expression plasmids including SEQ ID NO: 1, and the plasmid includes Eukaryotic PEAK series.

The present invention also involves mammalian cell lines, which contains the S protein gene of SARS-CoV virus that can express the fusion proteins of structural proteins of SARS-CoV virus. The mammalian cell lines include CHO, 293 and Vero cell lines and derived cell lines thereof.

The present invention also involved the methods for preparing the fusion proteins of SARS-CoV virus structural proteins, comprising the steps of:

(1) Transfecting a recombined expression plasmid which expresses a fusion proteins as claimed in claim 1 and endogenous dihydrofolate reductase (dhfr) and constructing mammalian expression cell lines;

(2) Producing over 10 μg of recombined proteins per million cells in the mammalian expression cell lines under normal growth circumstances in 24 hours; and

(3) Purifying the recombined proteins expressed in step (2).

In the method, the recombined expression plasmid has a leading sequence of fusion proteins, and such sequence is a leading sequence of CD5 protein. The coding genes of the structural proteins of the recombined expression plasmid are synthesized. The common or use bias codons sequence for human cells are used to replace the codons sequence of virus gene encoding the same amino acids to humanize and optimize the codons of the virus structural proteins. The gene of fusion protein expressing SARS-CoV virus structural proteins is synthesized as the S protein genes of SARS-CoV virus by the human common or use bias codons. The said gene of fusion protein is shown as SEQ ID NO: 1.

The screening drugs used in constructing mammalian expression cell lines preferably comprise puromycin and/or amethopterin

In the step of (2) of the method, preferably each million cells yield 30 μg or more recombined proteins in medium per 24 hours.

The present invention also involves the use of the fusion proteins for manufacturing vaccines for prophylaxis of SARS-CoV virus infection, and the use for producing kits for detecting SARS-CoV virus, the use for manufacturing or screening the medicaments for preventing from or treating the SARS-CoV virus infection, the use for preparing antibodies for preventing from the SARS-CoV virus infection.

The present invention in particular relates to arbitrary peptide fragments whose amino acids are removed, modified, or mutated from amino acid 318-510 in structural protein S of SARS-CoV virus, or to DNA sequences that express the arbitrary peptide fragments whose amino acids are removed, modified, or mutated from amino acid 318-510 in structural protein S of SARS-CoV virus, and the expressed amino acids.

The present invention further relates to the use of the DNA sequences or the amino acids expressed by the DNA sequences for preparing SARS-CoV virus vaccines, the said vaccines include DNA vector vaccines, protein vaccines, and virus vector vaccines.

### BRIEF DESCRIPTION OF THE DRAWINGS

FIG. 1 is the results of Western Blotting that detects and ensures the optimized fusion proteins of E, M, N, S expressed in host cells. From the left to right are E-Fc,M-Fc,N-Fc and S-Fc, respectively. The results confirm that the four structural proteins of SARS-CoV virus can all be well expressed in host cells.

FIG. 2 is an agarose gel electrophoresis analysis of S 1190 gene fragments-inserted expression vector digested by restricted enzymes. From left to right lanes are λ-Hind III Marker, S1190, DL2000 Marker respectively. λ-Hind III Marker from small to large (from bottom to top) are 564 bp (hard to distinguish in this figure), 2027 bp, 2322 bp, 4361 bp, 6557 bp, 9416 bp, 23130 bp; DL2000 Marker from small to large (from bottom to top) are 100 bp, 250 bp, 500 bp, 750 bp, 1000 bp, 2000 bp, the result indicates that S1190 gene fragments are well inserted into the expression vector.

FIG. 3 is the results of Western Blotting Western Blotting which detects and ensures the fusion protein of S1190-Fc expressed in host cells, indicates that S1190-Fc protein can be well expressed in host cells, and the size of the protein is about 185 KD.

FIG. 4 is the results of the polyacrylamide gel of the purified S1190-Fc protein stained with coomassie brilliant blue, and indicates that the resulted S1190-Fc protein is comparatively purity.

FIG. 5 is Cell-flow results of the Vero E6 cells combined with fusion protein S1190-Fc at 4° C. According to the results, the fusion protein S1190-Fc combines with Vero E6 cells. According to the published documentations, ACE2 exists as a receptor of S protein in the surface of the Vero E6 cells, and the binding area of S protein with ACE2 is amino acids 318-510. As a result, binding S1190-Fc fusion protein by this cell can detect the activity of the expressed S1190-Fc fusion protein. In the figure, the blank peak area is the negative control (PBS buffer), and area of the shadow is the results of experiment, indicates that S1190-Fc fusion protein binds Vero E6 cell.

FIG. 6 is the flow cytometry results of S1190-Fc fusion protein combining with 293E cells transfected with human ACE2 (hACE2). The 293E cells transfected with hACE2 bind to S1190-Fc protein at 4° C., then bind to anti-Fc antibodies tagged by FITC, non-transfected 293E cells bind to Fc antibodies as negative control (the blank peak area), then detected by flow cytometry. The results indicate that 293E cells transfected with hACE2 can bind to S1190-Fc protein, and thus result in an obvious shift (the shadow part).

FIG. 7 is the flow cytometry results of the 293E cells transfected with mice ACE2 (mACE2) combining with S1190-Fc protein. The 293E cells transfected with mACE2 combine with S1190-Fc protein at 4° C. followed by combining with anti-Fc antibodies tagged by FITC. The non-transfected 293E cells combine to Fc antibodies as negative control (blank peak area). Flow cytometry is used to detect the combination. The results indicate that 293E cells transfected with mACE2 can combine to S1190-Fc protein, and thus result in an obvious shift (the shadow part).

FIG. 8 is microphotographs of transfected 293ET cells. The left figure is a photograph of the cell fusion result of 293ET cells respectively transfected with ACE2 and S1190 gene (magnification, ×100). The right figure is a photograph of the 293ET cells without cell fusion after respectively transfected with CD4 and S1190 gene (magnification, ×100). The result indicates that protein S1190 can bind to ACE2 and cause cell fusion.

FIG. 9 is the results of co-immunoprecipitation (IP) of S1190-Fc with the receptor ACE2. Cells transfected respectively with S1190-Fc and ACE2, and the control cells transfected with control Fc and ACE2 are lysed and detected by using Western Blotting. The first lane is a lysate of cells transfected with Fc and ACE2 as control, the second lane is the IP results of lysate of cells transfected with control Fc and ACE2, the third lane is the lysate of cells transfected with S1190-Fc and ACE2 as control, the fourth lane is the IP results of lysate of cells transfected with S1190-Fc and ACE2, from the left to right, respectively. The result indicates that protein 1190 can bind to receptor ACE2, and Fc has no influence on the binding of S1190 to the receptor ACE2.

FIG. 10 is the results of down-regulation of the expression of the receptor ACE2 in cultured cells. Vero E6 cells are interacted completely with S1190-Fc protein respectively at 4° C. (blue line) and 37° C. (red line), at the same time Fc as control (black line) followed by detection with anti-Fc antibodies. The result indicates that at 37° C., S1190-Fc protein interacts with the receptor ACE2, and causes the down-regulation of the expression of the receptor ACE2.

FIG. 11 is the results of down-regulation of the expression of the receptor ACE2 in cultured cells. Vero E6 cells are interacted fully with S1190-Fc protein respectively at 4° C. (blue line) and 37° C. (red line), at the same time Fc as control (black line), followed by detection with anti-ACE2 antibodies. The result indicates that S1190-Fc fusion protein interacts with the receptor ACE2 at 37° C., and cause the down-regulation of the expression of the receptor ACE2.

FIG. 12 is lung elastance measurements of wild-type mice subjected to saline or acid perfusion and treated with S1190-Fc protein. Mice are divided into 4 groups, 5-7 mice each group, 2 groups are perfused with acid followed by treatment with S1190-Fc protein and control Fc, 2 groups are perfused with saline followed by treatment with protein S1190-Fc protein and control Fc in the same way. The dosage is 5.5 nmol/kg S1190-Fc protein or 5.5 nmol/kg control Fc for each mouse. The result indicates that there was a significant difference (p<0.05) of elastance between group of wild type mice perfused with acid accompanied with control Fc protein and group of wild type mice perfused with acid accompanied with S1190-Fc protein. Group of wild type mice perfused with acid accompanied with S1190-Fc protein has significantly higher magnitude of changes of elastance than groups perfused with acid accompanied with control Fc protein. It indicates that S1190-Fc protein can aggravate the acute lung injury of mice perfused with acid.

FIG. 13 is the pathological section of lung tissue of mice. The pathological sections are prepared by using the lung tissue of mice treated the same way as FIG. 11. The result coincides with the results of FIG. 11. Under the condition of acid perfusion, the lung of mice appears oedema resulting in acute lung injury, and the additional treating of S1190-Fc protein obviously aggravated the acute lung injury of the mice compared to the control Fc.

FIG. 14 is the score results about lung injury. This result confirms the results of FIG. 11 and FIG. 12, that under the condition of acid perfusion, acute lung injury is happened and the additional treatment of the fusion protein S1190-Fc obviously aggravated the acute lung injury of the mice compared to the control Fc. There is a significant difference between control Fc and the fusion protein S1190-Fc (p<0.01).

FIG. 15 is the results of wet-to-dry lung weight ratios. This result confirms the results of FIGS. 11, 12 and 13 that under the condition of acute lung injury induced by acid perfusion, the lung oedema resulted by treating with S1190-Fc protein is severer, the wet-to-dry lung weight ratio is larger than of group of control Fc. There is a significant difference between experimental group and control group (p<0.05).

FIG. 16 is the results of lung elastance measurements of wild-type mice perfused with acid or saline, and then with S1190-Fc protein or S318-510-Fc. The mice are divided into 5 groups, each group has 5-7 mice, 3 groups of them are perfused with acid, then respectively treated with the fusion protein S1190-Fc, fusion protein S318-510-Fc and control Fc, 2 groups of them are perfused with saline, then respectively treated with fusion protein S318-510-Fc and control Fc. The dosage for each mouse is 5.5 nmol/kg fusion protein or control Fc. The result indicates that there were significant differences of lung elastance measurements in all the measure time between the wild-type mice perfused with acid and control Fc and the wide-type mice perfused with acid and the fusion protein S1190-Fc, or with S318-510-Fc (p<0.05). It indicates that under the condition of acid perfusion, the fusion protein S1190-Fc and S318-510-Fc both can aggravate the acute lung injury.

FIG. 17 is the result of lung elastance measurements in Ace2 knockout mice perfused with acid or saline, and then with the fusion protein S1190-Fc. The process and group can be referred to the legend of FIG. 11. The result indicates that there is no significant difference of the influence on lung elastance measurements between the fusion protein S1190-Fc and the control Fc in Ace2 knockout mice when perfused with acid.

FIG. 18 is the result of the fusion protein S1190-Fc detected in lung homogenate after the protein intraperitoneally local injected. Fusion protein S1190-Fc is detected by pull-down assay with Protein G Sepharose and Western blotting with human anti-Fc specific antibody, while Fc is not detected in control groups.

FIG. 19 is the result of lung immunohistochemistry used for detecting fusion protein S1190-Fc with a human Fc-specific antibody. It indicates that the fusion protein S1190-Fc accumulates in bronchial epithelial cells (left panel; magnification, ×100), inflammatory exudates cells (middle panel; magnification ×200), and alveolar cells (right panel; magnification, ×200), which are the prone sites of acute lung injury.

FIG. 20 is the result of ACE2 protein falling in expression in the lungs of mice treated with fusion protein S1190-Fc. Lung homogenates were prepared from control Fc- and fusion protein S1190-Fc-treated wild-type mice and analyzed by western blot with ACE2-specific antibody. The result indicates S1190-Fc-treating causes the decreased expression of ACE2 protein in mice.

FIG. 21 is the result of level of AngII peptide in lungs of wild-type mice. Fusion protein S1190-Fc or control-Fc protein-treated wild-type mice are perfused with saline or acid followed by AngII level are determined at 3 hours by using enzyme immunoassay. The result indicates that there is significant difference (P<0.05) of AngII peptide level between fusion protein S1190-Fc- and control-Fc-treated wild-type mice perfused with acid, and the AngII peptide level of S1190-Fc-protein treated wild-type mice which are perfused with acid is significantly increased, much higher than the AngII peptide level of control-Fc protein-treated wild-type mice received acid perfusion.

FIG. 22 are the titers of neutralizing antibody in mice after immunized by fusion protein S1190-Fc protein (orange). Five-week old female balb/c mice are divided into 2 groups, each group has 5 mice. Group 1 immunized by injection of 50 μg fusion protein S1190-Fc with adjuvant per mouse at week 0, 2, 4, respectively; group 2 injected same dosage of Fc protein as control (blue). Sera are harvested at the 6^{th} week. Microquantity neutralization analysis is used to detect the existence of neutralizing antibody of the heat-inactive sera, which indicates that fusion protein S1190-Fc immunized mice can produce plentiful effective neutralizing antibodies, which can effectively prevent from SARS-CoV virus infection.

FIG. 23 is the results of agarose gel electrophoresis of S gene and fragments thereof inserted expression vector. The lanes are λ-Hind III Marker, S317, S318-510, S318-1190, S511-1190, S685, S900, S1148, S1190, DL2000 Marker from left to right, respectively. The bands of λ-Hind III Marker from small to large (from bottom to top) are 564 bp (hard to distinguish in this figure), 2027 bp, 2322 bp, 4361 bp, 6557 bp, 9416 bp, 23130 bp and the bands of DL2000 Marker from small to large (from bottom to top) are 100 bp, 250 bp, 500 bp, 750 bp, 1000 bp, 2000 bp. The result indicates that S protein gene fragments are already inserted into the expression vector.

FIG. 24 is the result of Western Blotting which detects and confirms the optimized S fusion protein and its truncated forms expressed in host cells, lanes 1-10 are S1190-Fc, about 185 KD; S1148 Fc, about 180 KD; S900 Fc, about 175 KD; S318-1190 Fc, about 160 KD; S511-1190 Fc, about 155KD; S685 Fc, about 155KD; S511 Fc about 140KD; S681-1190 Fc, about 120KD; S317 Fc, about 85 KD; S318-510-Fc, about 67 KD, respectively. This figure indicates that optimized S fusion protein and its truncated forms can be well expressed in host cells, while the wide-type sequence of it can hardly be expressed in mammalian cells; and shows that the optimization of expression is effective and feasible.

FIG. 25 is a photograph of non-cell-fusion of cells transfected with S317-Fc and ACE2 gene, transfected with gp120 and ACE2 gene respectively (magnification, ×100).

FIG. 26 is photographs of the cell fusion of cells respectively transfected with S318-510-Fc and ACE2 gene (magnification, ×100), S1190-Fc and ACE2 gene (magnification, ×100).

FIG. 27 is the photographs of the cell fusion of cells respectively transfected with fusion protein S511-1190 Fc and ACE2 gene (magnification, ×100), S681-1190 Fc and ACE2 gene (magnification, ×100).

A plurality of truncated forms of S protein and receptor ACE2, or gp120 (the surface protein of HIV) and receptor ACE2 are transfected into 293 cells, then the two kinds of cells are mixed post-transfection at 24 hours and photographs are taken at 48 hours. The above 6 photographs indicate that, ACE2 is the specific receptor of SARS-CoV virus, and the part that binds with ACE2 and cause cell fusion is S318-510, i.e., the 318^{th} to 510^{th} amino acids of S protein.

The present invention will be illuminated in details with the figures. As indicated, the present invention provides a S protein gene sequence of SARS-CoV virus which can be expressed in mammalian cell lines, e.g. said SEQ ID NO. 1 sequence of the present invention.

In addition, the present invention also provides a recombinant expression plasmid comprising SEQ ID NO. 1, preferably, the recombinant expression plasmid preferably comprises eukaryotic PEAK series.

The present invention also provides a fusion protein of SARS-CoV virus structural proteins, which can be expressed in S protein gene sequence of SARS-CoV virus in mammalian cell lines and which has the structure of X-Y-Z, wherein

X comprises the structural protein S, M, E or N of SARS-CoV virus, or any truncated forms of the above structural proteins.

Y is a linking part consisting of 0 to 20 of any amino acids.

Z is a Fc, its variants of human IgG₁ including hinge region, CH₂ and CH₃ domain or protein tags.

Thus mammalian expression cell lines can be constructed by transfecting a recombinant plasmid capable of expressing structural proteins of SARS-CoV virus and any truncated forms thereof and endogenous dihydrofolate reductase (dhfr).

The recombinant plasmid uses mammalian eukaryotic vectors having strong expression ability. Adoption of a stronger promoter to initiate the expression of genes successfully results in high level of expression of structural proteins of SARS-CoV virus and truncated forms thereof in mammalian expression system. The expression vectors of mammalian eukaryotic cell comprises PEAK series vectors, such as pEAK10, pEAK12, pEAK13 etc; the pCDNA series: pCDNA3.0, pCDNA4.0; the pCDM series: pCDM7, pCDM8, pCDM10, pCDM12; the preferable expression vector of eukaryotic cells is pEAK13. The promoters are selected from such as CMV, EF1α, CoYMV, CMV enhancer+chicken albumin promoter, SV40 promoter+enhancer. The preferable promoter is CMV enhancer+chicken albumin promoter.

The secretary sequence in the front of the interested protein sequences of the present invention is substituted with the known strong leading sequences of CD5L protein (CD5L) to promote the secretary expression of the interested proteins.

In the invention, secretary expression vector is used to effectively separate the expressed viral protein from host protein and DNA and the original secretary sequence of the wild SARS-CoV structural protein gene is removed and replaced by a piece of more powerful leader sequence, CD5L, which contains splicing signal. After translated into protein, it becomes a signal peptide for leading protein across the membrane and is responsible for leading the viral structural protein to go through the cell membrane to secret out of cell into medium so that the protein of interest could be separated from host proteins and DNA effectively. The method of the present invention makes protein purification process simpler and easier and decreases the difficulties of the protein purification as well as the probability of protein denaturation during protein purifying process. The signal peptide translated by the leading sequence can be cleavable due to the function of protein cleavage enzyme, so the structure of the viral protein is not affected. CD5L sequence replaces the original secretary sequence of the wild SARS-CoV structural protein gene, and the sequence is shown as follows:

5′ATGCCCATGGGGTCTCTGCAACCGCTGGCCACCTTGTACCTGCTGGGG
ATGCTGGTCGCTTCCTGCCTCGGAGCG 3′.

In the present invention, artificial synthesis is conducted on coding gene of structural protein of the plasmid for expressing fusion proteins of structural proteins of SARS-CoV virus by replacing codons coding identical amino acids in the viral gene with conventional (bias) codons in human cells, thereby human coding optimization of structural protein gene of virus is well carried out.

To increase the expression level of SARS-CoV structural proteins and truncated forms thereof in mammalian expression system, gene optimization is adopted in the present invention. Said gene optimization includes codon humanization and optimization.

The codon humanization refers to replacing rarely used codons in human cells with frequently used bias codons, since inequality and bias codon use to various extent are common in many organisms, and in the present invention, human cells are adopted as the hosts and the object is also for application to human bodies. Therefore rarely used codons in human body are replaced by use bias codons that frequently are used in human body.

Codon optimization, as a method for gene optimization, refers to replacing rarely used codons in gene coding of fusion protein with frequently used codons in the expression hosts. The amino acids sequence of S-protein of wild SARS-Cov is available in Genebank, when codon of each amino acid is replaced by a more frequently used one in human host cells, a plurality of optimized gene sequences are obtained resulting in enhanced expression level of protein.

In the invention, those codons rarely used in human cells are replaced by high performance codons of human cells encoding the same amino acids, for example, the codon GGC of Gly is chosen to replace other codons (GGA/GGT/GGG), GAG of Glu replaces GAA, and GAC of Asp replaces GAT, etc.

To be clearer, a list is given to show the usage frequency of codons in human highly expressed genes, according to the usage frequency of codons. In the invention, codon replacement is shown in the following table according to the ratio of usage frequency. The more frequently used codons are selected to ones in correspondence to amino acids to enhance the expression of interested genes. The usage frequency ratio of codons in highly expressed genes of human is shown as follows:

| Amino acids | codons | Number | /1000 | Rate |
|---|---|---|---|---|
| Gly | GGG | 905.00 | 18.70 | 0.24 |
| Gly | GGA | 527.00 | 10.89 | 0.14 |
| Gly | GGT | 443.00 | 9.15 | 0.12 |
| Gly | GGC | 1868.00 | 38.60 | 0.50 |
| Glu | GAG | 2422.00 | 50.05 | 0.75 |
| Glu | GAA | 801.00 | 16.55 | 0.25 |
| Asp | GAT | 595.00 | 12.30 | 0.25 |
| Asp | GAC | 1827.00 | 37.76 | 0.75 |
| Val | GTG | 1867.00 | 38.58 | 0.64 |
| Val | GTA | 135.00 | 2.79 | 0.05 |
| Val | GTT | 202.00 | 4.17 | 0.07 |
| Val | GTC | 732.00 | 15.13 | 0.25 |
| Ala | GCG | 653.00 | 13.49 | 0.17 |
| Ala | GCA | 491.00 | 10.15 | 0.13 |
| Ala | GCT | 655.00 | 13.54 | 0.17 |
| Ala | GCC | 2059.00 | 42.55 | 0.53 |
| Arg | AGG | 512.00 | 10.58 | 0.18 |
| Arg | AGA | 302.00 | 6.24 | 0.10 |
| Ser | AGT | 357.00 | 7.38 | 0.10 |
| Ser | AGC | 1172.00 | 24.22 | 0.34 |
| Lys | AAG | 2125.00 | 43.91 | 0.82 |
| Lys | AAA | 481.00 | 9.94 | 0.18 |
| Asn | AAT | 324.00 | 6.70 | 0.22 |
| Asn | AAC | 1122.00 | 23.19 | 0.78 |
| Met | ATG | 1078.00 | 22.28 | 1.00 |
| Ile | ATA | 90.00 | 1.86 | 0.05 |
| Ile | ATT | 319.00 | 6.59 | 0.18 |
| Ile | ATC | 1374.00 | 28.39 | 0.77 |
| Thr | ACG | 405.00 | 8.37 | 0.15 |
| Thr | ACA | 378.00 | 7.81 | 0.14 |
| Thr | ACT | 362.00 | 7.48 | 0.14 |
| Thr | ACC | 1504.00 | 31.08 | 0.57 |
| Trp | TGG | 653.00 | 13.49 | 1.00 |
| End | TGA | 109.00 | 2.25 | 0.55 |
| Cys | TGT | 326.00 | 6.74 | 0.32 |
| Cys | TGC | 707.00 | 14.61 | 0.68 |
| End | TAG | 43.00 | 0.89 | 0.22 |
| End | TAA | 46.00 | 0.95 | 0.23 |
| Tyr | TAT | 362.00 | 7.48 | 0.26 |
| Tyr | TAC | 1042.00 | 21.53 | 0.74 |
| Leu | TTG | 316.00 | 6.53 | 0.06 |
| Leu | TTA | 78.00 | 1.61 | 0.02 |
| Phe | TTT | 337.00 | 6.96 | 0.20 |
| Phe | TTC | 1378.00 | 28.48 | 0.80 |
| Ser | TCG | 325.00 | 6.72 | 0.09 |
| Ser | TCA | 167.00 | 3.45 | 0.05 |
| Ser | TCT | 453.00 | 9.36 | 0.13 |
| Ser | TCC | 958.00 | 19.80 | 0.28 |
| Arg | CGG | 611.00 | 12.63 | 0.21 |
| Arg | CGA | 184.00 | 3.80 | 0.06 |
| Arg | CGT | 211.00 | 4.36 | 0.07 |
| Arg | CGC | 1086.00 | 22.44 | 0.37 |
| Gln | CAG | 2023.00 | 41.81 | 0.88 |
| Gln | CAA | 289.00 | 5.97 | 0.13 |
| His | CAT | 237.00 | 4.90 | 0.21 |
| His | CAC | 871.00 | 18.00 | 0.79 |
| Leu | CTG | 2885.00 | 59.62 | 0.58 |
| Leu | CTA | 167.00 | 3.45 | 0.03 |
| Leu | CTT | 242.00 | 5.00 | 0.05 |
| Leu | CTC | 1278.00 | 26.41 | 0.26 |
| Pro | CCG | 482.00 | 9.96 | 0.17 |
| Pro | CCA | 457.00 | 9.44 | 0.16 |
| Pro | CCT | 569.00 | 11.76 | 0.19 |
| Pro | CCC | 1410.00 | 29.14 | 0.48 |

In the invention, several optimized DNA sequences with gene coding optimization had been obtained, wherein the preferable synthesized S-protein gene sequence of SARS-CoV is shown in SEQ ID NO. 1.

According to the present invention, the eukaryotic cell lines for recombine plasmid transfection are selected from CHO, 293, Vero and derivative cells thereof.

Expression cell lines are constructed with appropriate host expression cell lines with high-level expression such as 293 cell, CHO cell or Vero cell and derivatives thereof. Anti-puromycin gene contained in the recombinant plasmid are utilized to screen and transfect S-protein gene and truncated forms thereof followed by ELISA or Western Blotting for quantification and qualification so that the optimal expression cell line can be acquired wherein, 293E, 293ET and CHO cells have relatively high expression level, CHO cell in particular. Habituated culture is carried out on cell lines with high and stable expression for further improving the expression of viral proteins in order to pave a way for batch preparation and industrialized production.

Those mammalian expression cell lines involved in the invention has been deposited in China General Microbiological Culture Collection Center (CGMCC) since Jul. 6, 2005, with the deposition Number. of 1408, 1409 and 1410 respectively.

In the invention, puromycin is used as the screening drug in construction of eukaryotic cell expression lines and methotrexate is used for improving protein expression level in cells.

In the invention, puromycin-resistant gene is inserted into the recombinant plasmid constructed. Puromycin is an antibiotic that can kill eukaryotic cells. When cells are transfected into recombinant plasmid with puromycin-resistant gene, the resistance to puromycin can be improved. Mammalian cells with successful transfection can be screened with the puromycin resistance difference between cells with and without puromycin-resistant genes by gradient addition of puromycin into the cell medium. This recombinant plasmid also carries endogenous dihydrofolate reductase (dhfr) gene, therefore methotrexate can be used for cell habituation and improvement of protein expression level.

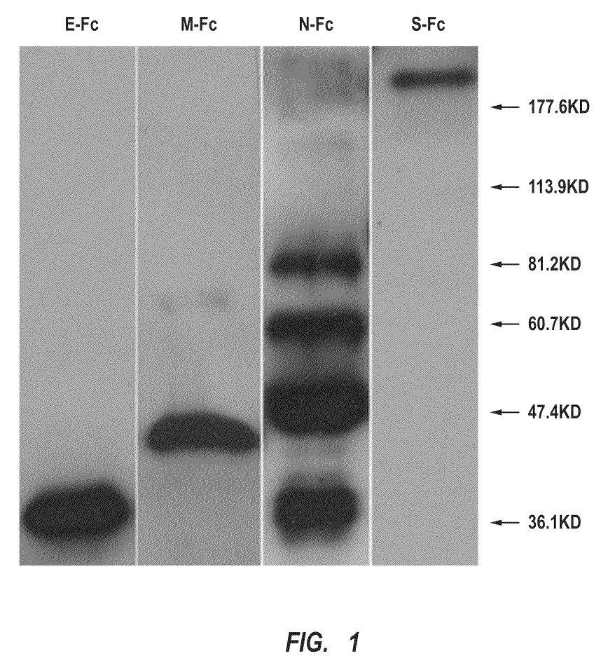

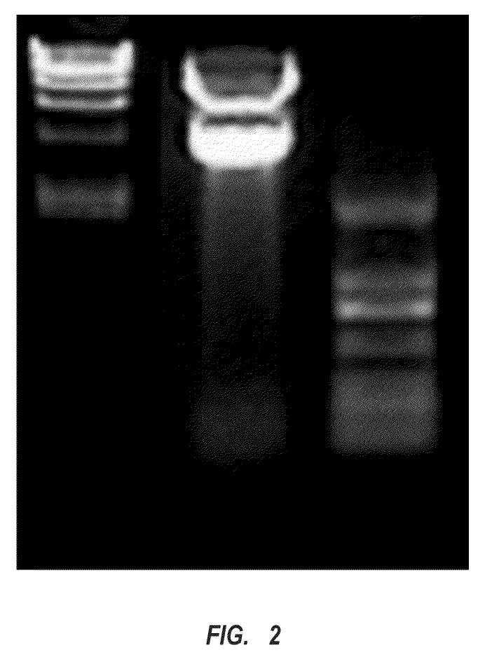

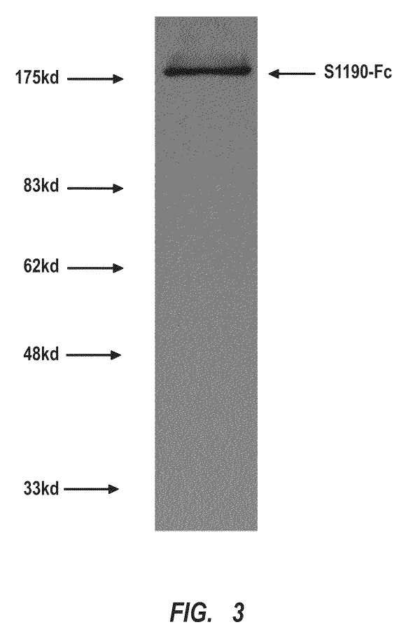

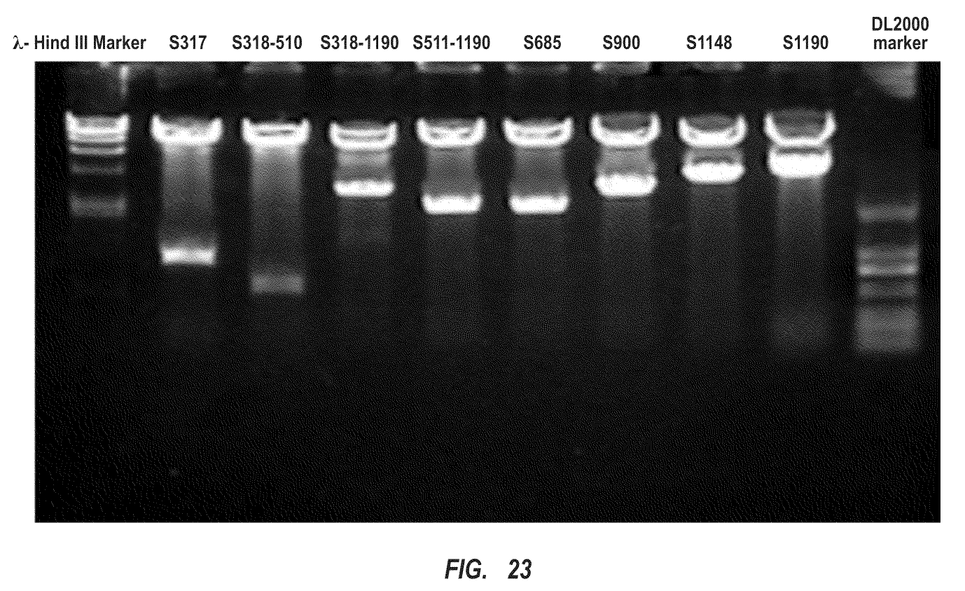

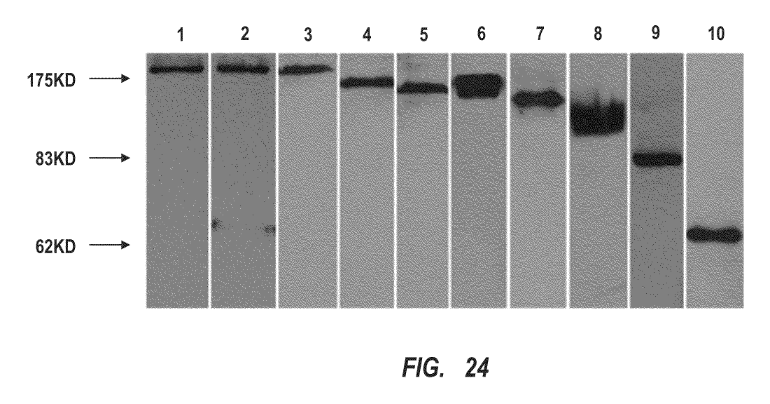

The fusion proteins of full length SARS-CoV structural proteins expressed in the invention (E-Fc, M-Fc, N-Fc, S-Fc) is given in FIG. 1. The S protein of SARS-CoV and all the truncated formed fusion proteins thereof (317-Fc, 511-Fc, 685-Fc, 900-Fc, 1148-Fc, 1190-Fc, 318-510-Fc, 318-1190-Fc, 511-1190-Fc and 681-1190-Fc) are given in FIGS. 23 and 24.

Construction of cell expression lines avoids the instability of transient transfection state, and screening superior expression lines can increase the production. The expression can be further improved by cell habituation etc., thereby realizing the batch preparation and industrialized production of proteins.

Example 1 and 2 show the process of the plasmid construction, and Example 3 shows the process of cell line construction.

(2) In normal cell growth state, over 10 μg recombinant protein can be harvested from medium per 106 cells at 24 hours. Cells are counted and then cultured. After three days, the medium which cells have grown in are collected and tested by ELISA to detect the expression level; and then expression level of S protein and the truncated form series thereof are obtained by computation. According to the methods disclosed in the invention, the fusion protein with high expression of various SARS-CoV structural protein and truncated forms thereof are obtained. And the expression level is over 10 μg/106 cells/24 hours (extracted from cell medium). Wherein, the production of S1190-Fc (full-length S protein without the transmembrane domain) is over 10 μg/106 cells/24 hours, and of truncated form of S protein (S318-510-Fc) is over 30 μg/106 cells/24 hours.

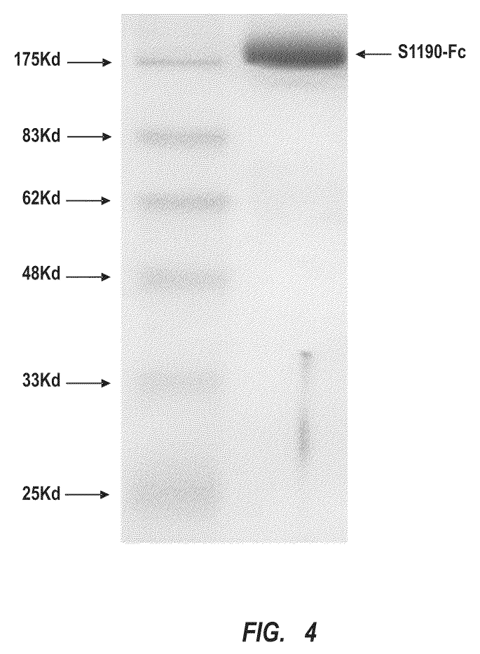

(3) Proteins gained in step (2) are then purified. Secretary expression simplifies the protein purification and reduces the possibility of denaturation in purification; more, effective separation can be executed from host proteins and DNA. After the protein of interest is purified by affinity column chromatography and molecular sieve, its purity can exceed 99% (see FIG. 4), which is confirmed by HPLC-MS analysis.

Detailed steps are described in Example 10.

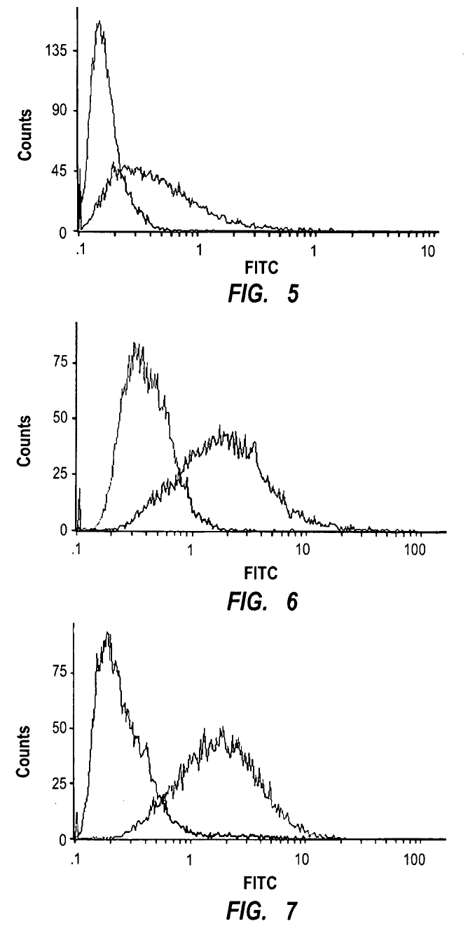

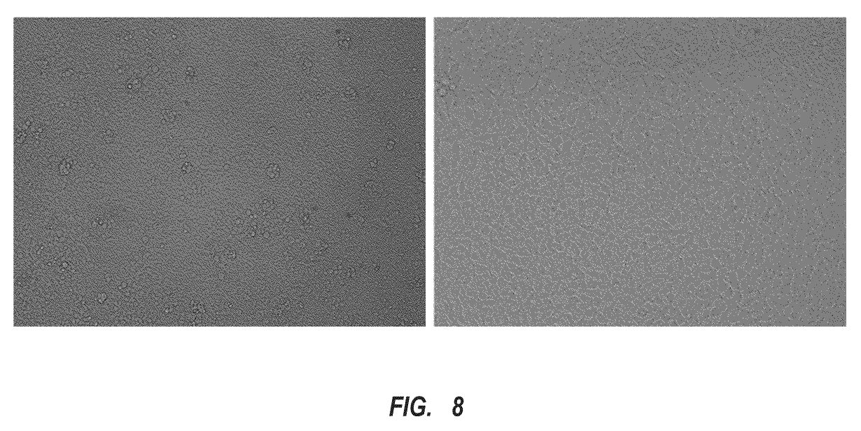

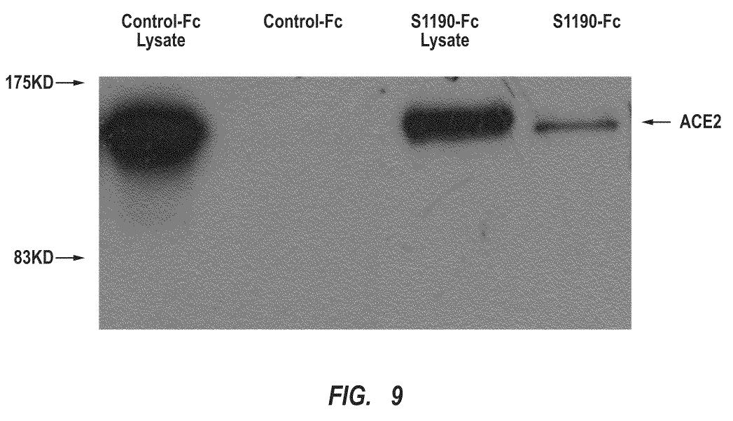

The proteins expressed and purified in the invention have corresponding biological activity in vivo. For instance, S1190-Fc expressed and purified in the invention can bind to ACE2, receptor of S protein (FIGS. 5, 6, 7 and 9). S1190-Fc can also fuse with and subsequently enter into cells carrying receptor thereof (FIGS. 8, 10 and 11).

The proteins expressed and purified in the invention can be used to develop vaccines for preventing SARS-CoV infection.

By successive optimization of S protein, expression of S protein and a series of truncated forms thereof in the invention had been completed and studied on function of each region to have a lot of results and data. The study results hold a promise for the application of S protein and the short form series thereof. Furthermore, the interaction between S protein and ACE2 both in vivo and in vitro is studied and the result indicates that binding of S protein can result in down-regulation of ACE2 expression, which can aggravate the severity of acute lung injury. In the present invention, further tests confirm that the sites substantially responsible for binding to ACE2 and down-regulating ACE2 are the 318th to 510th of S protein. So in vaccine preparation, S318-510 should be removed or S protein shall be mutated or modified for restriction or prohibition of combination ability with ACE2; especially the S318-510 in S protein should be mutated or modified to prevent pathological response induction. So proper immunogen that induce cellular immunity, humoral immunity and effective neutralizing antibodies can be used to prepare a vaccine for preventing from SARS-CoV infection.

Mice immunized with the fusion protein expressed and purified in the invention can produce efficient neutralizing antibodies against SARS-CoV virus.

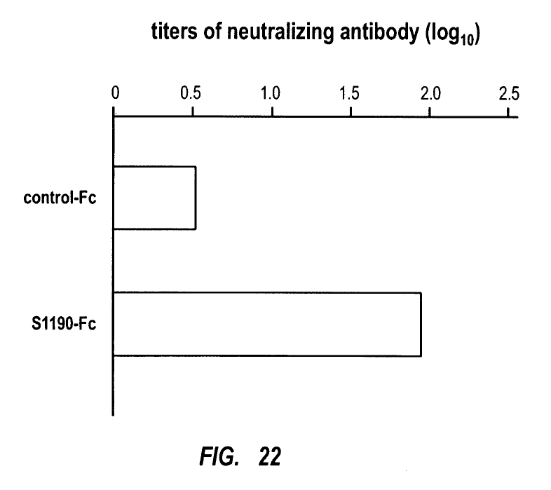

Five female balb/c mice (five-week old) in each of two groups are immunized: one group is immunized with 50 μg S1190-Fc with adjuvants at 0, 2, 4 weeks, while the other group is injected with the same dose Fc with adjuvants as control. The sera sampled from the mice two weeks after the third immunization are heat-inactivated and then microneutralization assay is performed to detect the existence of neutralization antibodies. The result is positive and micro neutralization assay is performed to analyze antibody titers of twofold dilution heat-inactivated sera dilution. Neutralizing antibodies are added to a 96-well plate with every three wells for a concentration gradient. Then SARS-CoV is added to each well at dose of 100×TCID50 (Vero E6 monolayer cells) and cytopathic effect (CPE) is observed on the third and fourth day. At last the titer of neutralizing antibodies is obtained by calculation on the concentration gradient that can inhibit CPE completely in half of the wells with RM formula. The result indicates that the difference between S1190-Fc group and the control group is significant. Mice immunized with S1190-Fc can produce a great deal of neutralizing antibodies that can prevent SARS-CoV infection effectively. Detailed steps are shown in Example 11. And the results are shown in FIG. 22. The invention indicates that mice immunized with any fusion protein of truncated form of S protein can produce neutralizing antibodies of different titers against SARS-CoV, preventing from SARS-CoV infection to different extents.

The fusion proteins disclosed in the invention can be used for preparation of virus detection kits.

The virus structural protein and truncated forms thereof provided by the present invention can be used as a new reagent for detecting SARS-CoV; through existence test of corresponding antibody in blood, the SARS-CoV virus infection possibility can be confirmed. Animal test done in the invention indicates that S protein has strong immunogenicity and therefore can be used as an immunodiagnostic antigen for corresponding antibody detection in blood. Protein, which is expressed in mammalian host cells and which has antigenicity and can react with corresponding SARS-CoV resistant antibodies, can be purified and linked to an enzyme-label plate for forming a detection kit according to related principles of ELISA. If a human body is infected with SARS-CoV, the corresponding antibodies produced in blood can absorb and connect with the S-protein of the enzyme-label plate, and after further reaction of marked antibodies, will appear positive and be detected for diagnosis assistance.

Also, said fusion protein can be used to develop or screen SARS-CoV resistant drugs.

S protein disclosed in the invention can be used for therapeutic drug screening. The pathogenic mechanism of SARS-CoV lies in the interaction between S protein and receptor ACE2. Thus, drugs including small-molecule compounds, polypeptides and genetic engineering drugs which can inhibit the interaction between S protein and its receptor ACE2 shall be the objects in SARS-CoV resistant drug screening for inhibition of SARS-CoV to enter into interested cells. The invention has confirmed that mice injected with S1190 can produce a great deal of neutralizing antibodies, which can inhibit 100 fold TCID50 of SARS-CoV from infecting Vero E6 cells.

The fusion protein disclosed in the invention can be used to prepare antibodies against SARS-CoV infection.

S protein disclosed in the invention can be used to select monoclonal antibodies, especially humanized monoclonal antibodies, which can specifically bind to S protein, thereby preventing the interaction between S protein and receptor ACE2. Therefore, said monoclonal antibodies can be used as a therapeutic drug for SARS or be used to carry out passive immunoprotection in public.

Said amino acid sequence of SARS-CoV structural protein in the description is derived from GenBank NC_{—}004718.

The invention also relates to a DNA sequence in which any fragment or all of the 318th to 510th amino acids of SARS-CoV structural protein are removed, modified or mutated, and also to amino acids sequence expressed by the DNA sequence.

The invention also relates to the use of the DNA sequence or expressed amino acid thereof in preparation of vaccines for SARS-CoV prevention. Said vaccines include DNA vector vaccines, protein vaccines and virus vector vaccines.

Objects of both the DNA sequence and amino acids acquisition are to prohibit or restrict the binding of S protein of SARS-CoV structural proteins to receptor ACE2.

Acute respiratory distress syndrome (ARDS) is the most severe form of acute lung injuries and is characterized by pulmonary oedema due to increased vascular permeability, the accumulation of inflammatory cells and severe hypoxia. Predisposing factors for ARDS are diverse and include sepsis, aspiration, pneumonias and infections with the severe acute respiratory syndrome (SARS) coronavirus or avian influenza/human influenza. Test data of the invention show that acute lung injury including SARS-CoV infection in mice results in considerably reduced ACE2, a key enzyme in the renin-angiotensin system, which leads to imbalance of the renin-angiotensin system to the inclination of AngII increase. This newly found role of ACE2 and the renin-angiotensin system seem to be in no association with vasoconstriction but with vascular permeability regulation. Other metabolites of ACE2 like bradykinin may play important roles in vivo, however, as is proved by test in the invention, ACE2 functions substantially through AngII.

The rennin-angiotensin system (RAS) has an important role in maintaining blood pressure stability as well as fluid-salt balance. ACE2 is a homologue of ACE, and functions as a negative regulator of the rennin-angiotensin system. Interestingly enough, experimental SARS-CoV infection in vivo can lead to considerably reduction of ACE2 expression in mouse lungs. Although ACE2 is expressed in the lungs of humans and mice, nothing is known about its function in the lungs. To elucidate the role of ACE2 in acute lung injury as well as in lung failure, the effect of Ace2 gene deficiency in mice is determined in experimental models, which mimic the common lung pathological manifestations observed in a plurality of human diseases, including sepsis, acid aspiration and pneumonias such as SARS and avian influenza A.

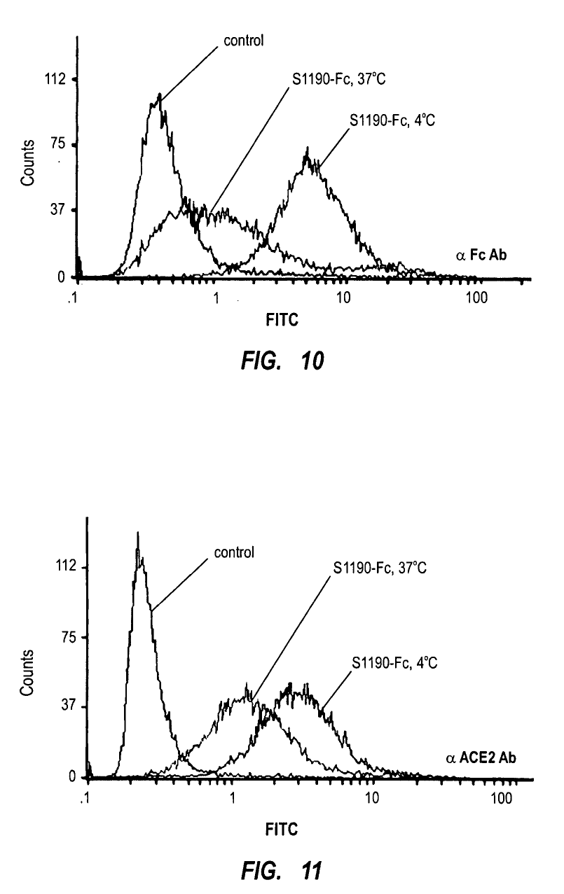

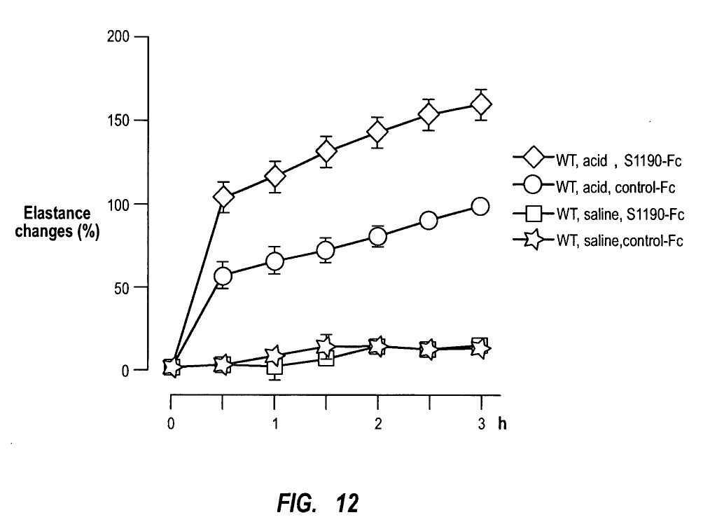

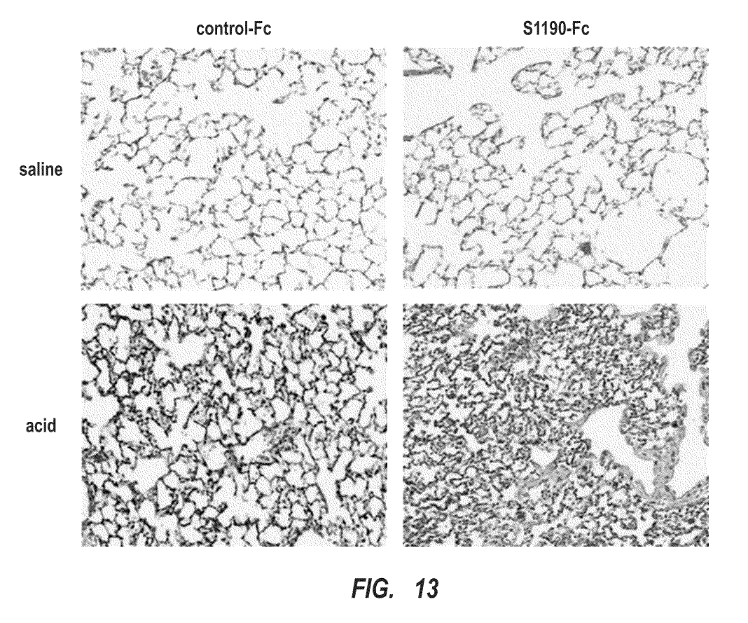

The inventor finds that binding of S protein to ACE2 can lead to down-regulation of ACE2 protein expression (see FIGS. 10, 11 and 20), which then causes or aggravates acute lung injury through the signal transmission path of RAS (see FIGS. 12, 13, 14, 15, 16, 17, 18, 19 and 21).

Tests of antibodies against Fc and cell line culture indicate down regulation of ACE2 expression. Vero E6 cells are made to react thoroughly with the fusion protein S1190-Fc at 4° C. (blue line) and 37° C. (red line), and meanwhile, Fc is used as the control (black line). The binding is detected by antibodies against Fc. The inventor finds that S1190-Fc protein binds to receptor ACE2 at 37° C. and leads to down regulation of ACE2 (FIG. 10).

Tests of antibodies against Fc and cell line culture indicate down regulation of ACE2 expression. Vero E6 cells are made to react thoroughly with S1190-Fc protein at 4° C. (blue line) and 37° C. (red line), and meanwhile, Fc is used as the control (black line). After detected by antibodies against Fc, the inventor finds that S1190-Fc protein binds to receptor ACE2 at 37° C. and leads to down regulation of ACE2 (FIG. 11).

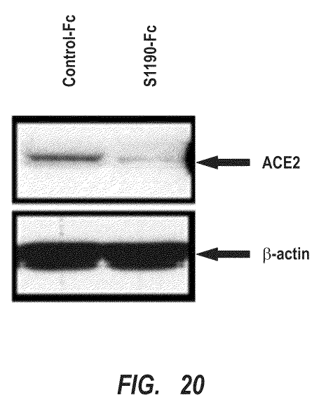

Wild-type mice treated by fusion protein S1190-Fc show a reduced ACE2 expression in the their lungs. Wild-type mice are treated respectively with fusion protein S1190-Fc and control-Fc protein, and then Western blotting is performed using ACE2 antibodies for detection. And the inventor finds that S1190-Fc treatment of wild-type mice results in reduced ACE2 expression in the lungs (FIG. 20).

Acid or saline instillation mixed with S1190-Fc in wild-type mice results in changes in lung elastance. Mice are divided into four groups, two groups of WT mice (n=5-7 per group) instilled with acid, in which one group plus S1190-Fc (5.5 nmol/kg), the other plus control-Fc (5.5 nmol/kg); and another two groups of WT mice (n=5-7 per group) instilled with saline, in which one group plus S1190-Fc (5.5 nmol/kg), the other plus control-Fc (5.5 nmol/kg). And the results indicate that there is a significant difference (P<0.05) between acid- and S 1190-Fc-treated WT mice and acid- and control-Fc-treated WT mice over the whole time course, lung elastance change of the former group is significantly greater in degree than that of the later (see FIG. 12). The inventor finds that with acid perfusion, S1190-Fc treatment aggravates acute lung injury.

FIG. 13 is a tissue pathological section of mouse lung. The pathological sections made from the above acid-treated WT mice match with the illustration in FIG. 12. Acid treatment results in significant pulmonary oedema and acute lung injury and S1190-Fc treatment worsens acute lung injury compared with the control-Fc group.

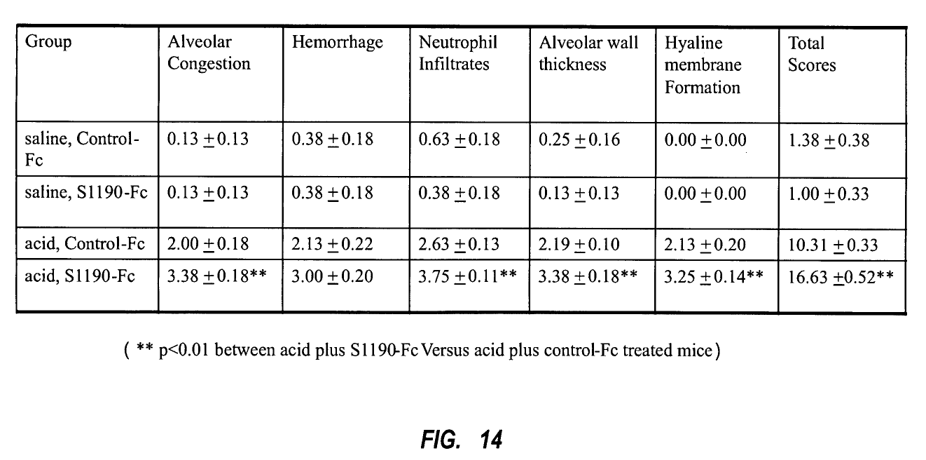

FIG. 14 is the result of lung injury measurements, which confirms the results of FIGS. 12 and 13 that acid treatment results in acute lung injury and S1190-Fc treatment worsens the acute lung injury compared with the control-Fc group with a significant difference between (p<0.01).

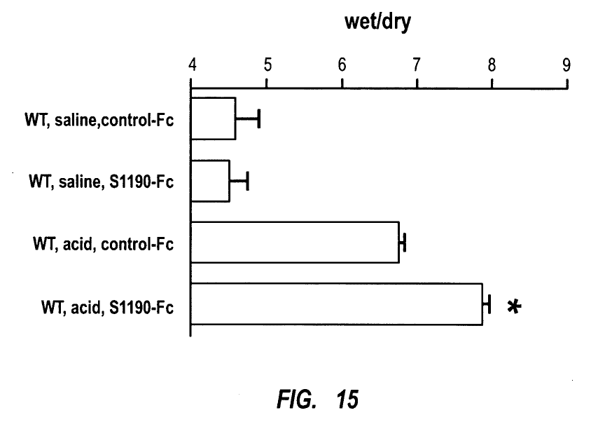

FIG. 15 is the result of wet-to-dry lung weight ratio. This result confirms the results of FIGS. 12, 13 and 14 that S1190-Fc treatment worsens oedema of the acid -induced acute lung injury and showed a greater wet-to-dry lung weight ratio, compared with control-Fc group with a significant difference between (p<0.05).

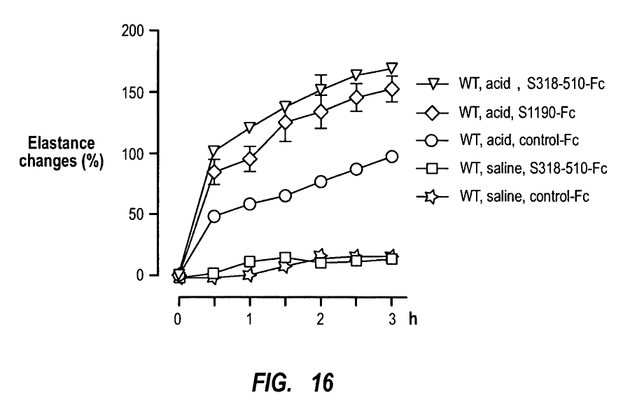

Acid or saline instillation mixed with S1190-Fc or S318-510-Fc in wild-type mice resulted in changes in lung elastance. Among five groups (n=5-7 per group), three groups of WT mice are instilled with acid, one group plus fusion protein S1190-Fc (5.5 nmol/kg), the second group plus fusion protein S318-510-Fc (5.5 nmol/kg) and the third group plus control-Fc (5.5 nmol/kg); another two groups of WT mice are instilled with saline, one group plus fusion protein S318-510-Fc (5.5 nmol/kg), the other plus control-Fc (5.5 nmol/kg). And the results indicate that there is a significant difference (P<0.05) between acid- and fusion protein S1190-Fc-treated WT mice/ acid+S318-510-Fc-treated WT mice and acid- and control-Fc-treated WT mice over the whole time course; lung elastance change of the former group is significantly greater in degree than that of the later (see FIG. 16). The inventor finds that both fusion proteins S1190-Fc and S318-510-Fc treatment worsens acid-induced acute lung injury.

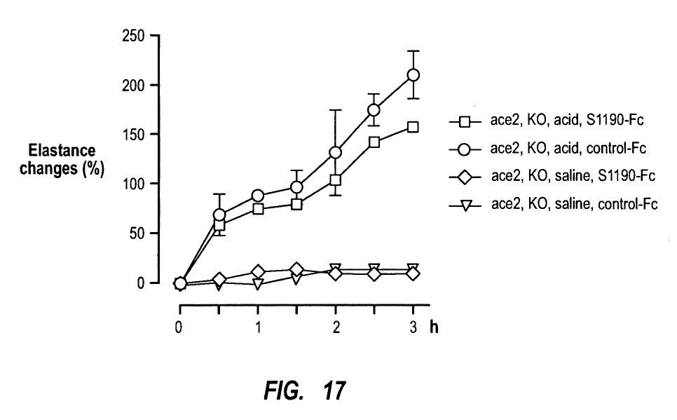

Acid or saline instillation mixed with fusion protein S1190-Fc in ACE2 knock out mice results in changes in lung elastance. The processing and grouping methods are similar to those described in FIG. 12. And the result indicates that there is no significant difference between the lung elastance of fusion protein S1190-Fc treatment group and the control-Fc treatment group in acid-treated ACE2 knock-out mice (FIG. 17), therefore it can be concluded that binding of fusion protein S1190-Fc to ACE2 causes or leads to acute lung injury.

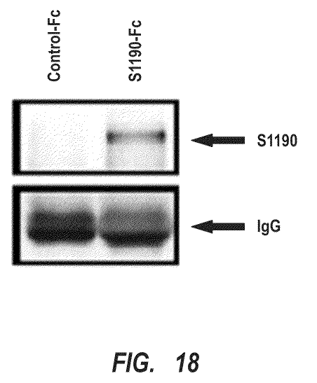

After partial intraperitoneal injection of fusion protein S1190-Fc, fusion protein S1190-Fc protein can detected in lung homogenate with Fc-specific antibodies by Western blotting and protein G agarose methods, whereas Fc is not detected in mice of the control group (FIG. 18).

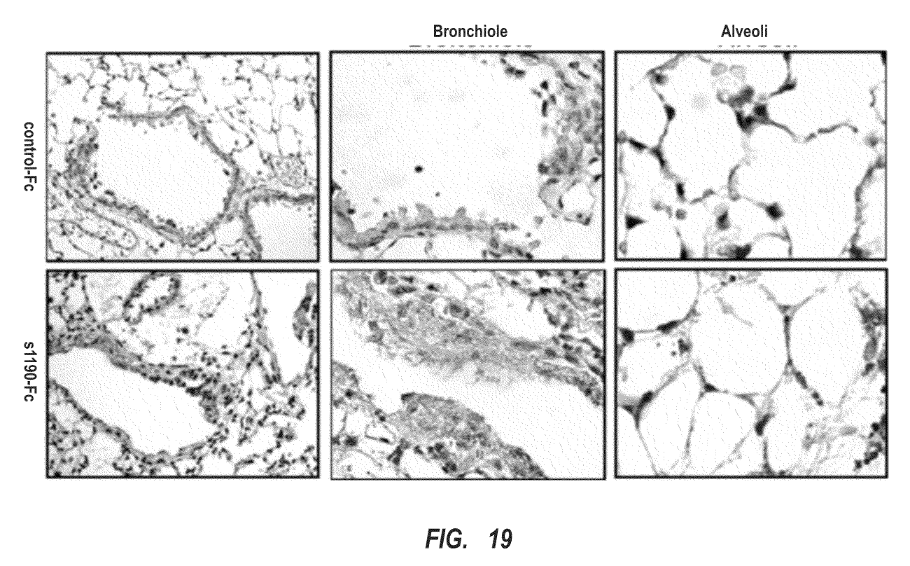

Localization of fusion protein S1190-Fc in mouse lungs is done. Results of immunohistochemical analysis indicate that fusion protein S1190-Fc is localized to bronchial epithelial cells (left, magnification ×100), inflammatory secretary cells (middle, magnification ×200) and alveolar cells (right, magnification ×200), which are the sites prone to suffer acute lung injury. In other words, fusion protein S1190-Fc is primarily localized to parts with acute lung injuries (FIG. 19).

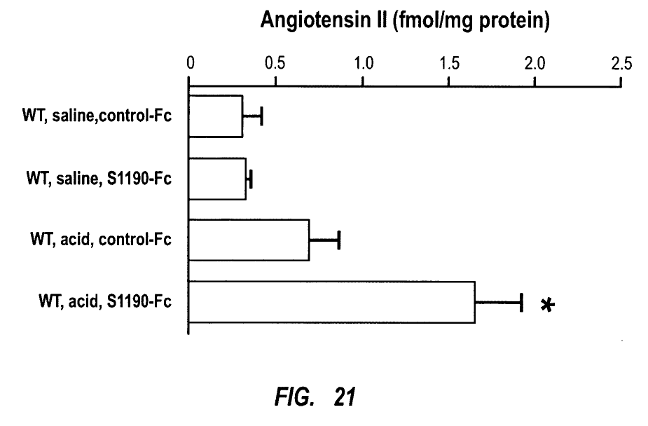

Influence of fusion protein S1190-Fc on AngII levels in the lung tissue of wild-type mice. After saline or acid perfusion, lungs of wild-type mice are treated with fusion protein S1190-Fc or control Fc. And AngII levels are determined after three hours by enzyme immunoassay (EIA). The result shows that there is a significant difference on AngII levels between fusion protein S1190-Fc- and control-Fc-treated wild-type mice after acid treatment (p<0.05). Acid treatment and fusion protein S1190-Fc treatment significantly increases AngII levels in the lungs of wild-type mice far higher than those of the group with acid treatment and control-Fc addition (FIG. 21).

All the above experiments confirm that S1190 protein triggers or worsens acute lung injury by binding to ACE2 and further down-regulating ACE2 expression and the ACE2 binding sites include the 318th to 510th amino acids of the S protein. The fragment (S318-510) itself can cause or aggravate acute lung injury and so shall be removed or modified in vaccine preparation.

Said truncated forms of SARA-CoV structural protein S comprises any truncated form the 318th to 510th amino acids are removed. According to published literatures, ACE2 is the receptor of SARA-CoV structural protein S. The invention, through in vitro and in vivo tests of interaction between S protein and ACE2, discovers that S protein can lead to down regulation of ACE2 and that the down regulation can aggravate acute lung injuries. The invention, through further tests, the dominating sites for binding to ACE2 and leading to down regulation of ACE2 are the 318^{th} to 510^{th} amino acids of the S protein. Therefore, in vaccine preparation, the amino acid sequence shall be removed or modified for preventing initiation of a series of pathological process; and vaccines can be prepared with screened truncated forms (the amino acid sequence has been removed or modified) of S protein which has appropriate immunogenicity and can initiate proper cell-mediated and humoral-mediated immunity reactions to produce valid neutralizing antibodies.

The affinity of said SARA-CoV structural protein S comprising mutated or modified S protein and any truncated forms thereof to ACE2 is weakened or lost. The invention confirms that down-regulation of ACE2 expression can aggravate acute lung injury and that the dominating sites for binding to ACE2 and leading to down regulation of ACE2 are the 318^{th} to 510^{th} amino acids of the S protein. So in vaccine preparation, mutation or modification shall be conducted on the S protein, in particular, the 318^{th} to 510^{th} amino acid sequence thereof, for elimination or reduction the affinity to receptor ACE2. Mutation and modification can keep excellent immunogenicity for initiation without induction of pathological injuries, and therefore, is an important method in gene engineering vaccine preparation.

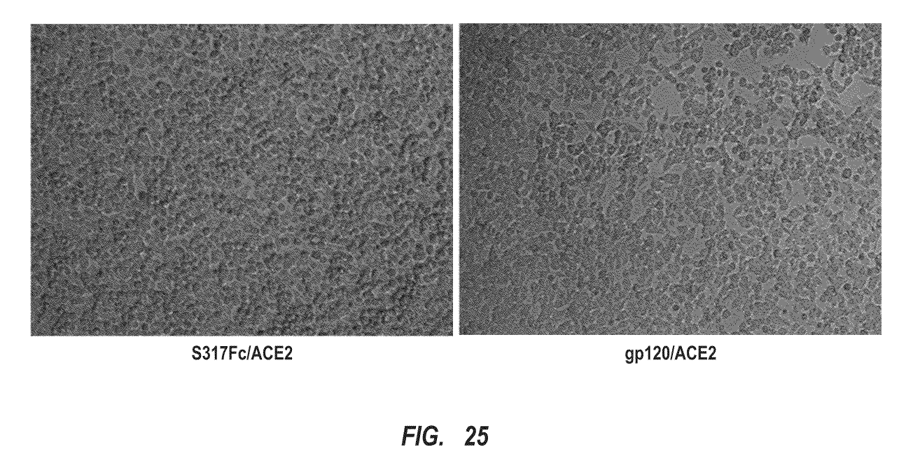

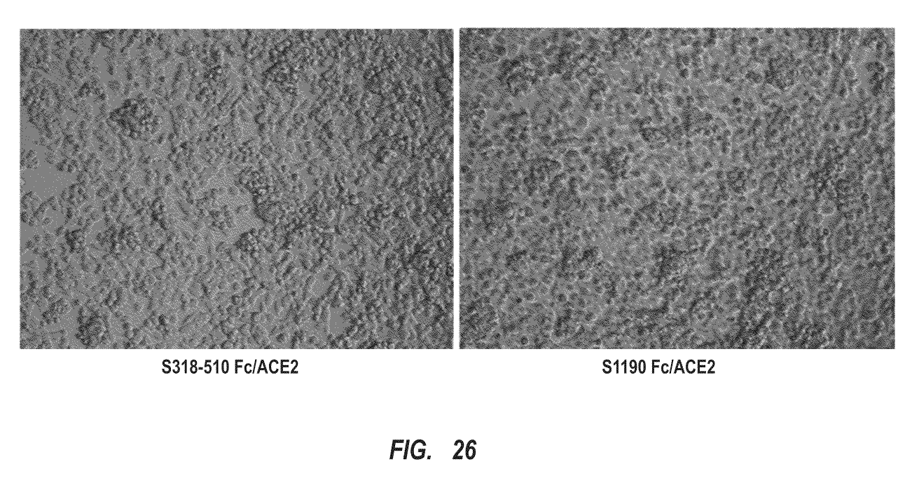

FIG. 25 is a picture of fusion protein S317-Fc and ACE2 transfected cells with no fusion between gp120 and ACE2 (magnification ×100).

FIG. 26 is a picture of fusion protein S318-510-Fc transfected cells and ACE2 wherein fusion protein S1190-Fc transfected cells and ACE2 are fused (magnification, ×100).

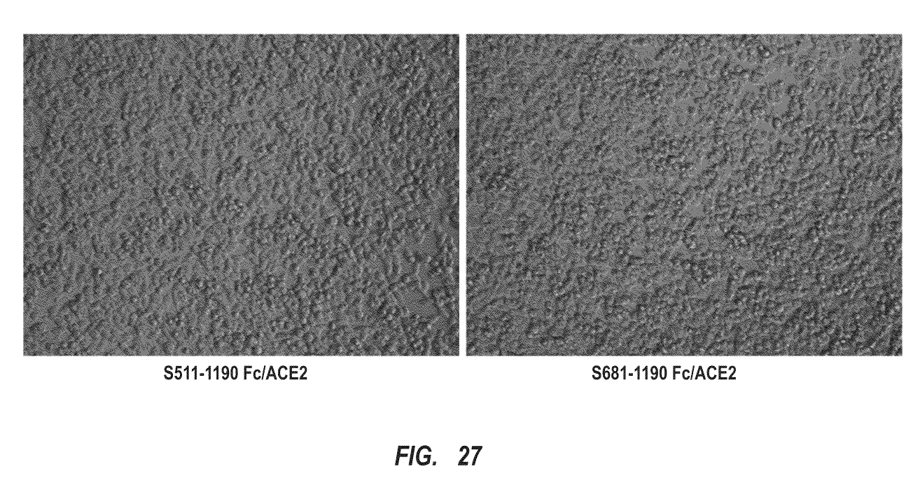

FIG. 27 is a picture of fusion protein S511-1190-Fc transfected cells, and ACE2, wherein S681-1190-Fc and ACE2 are not fused (magnification, ×100).

293 cells are respectively transfected with the above-mentioned truncated forms of S protein and ACE2 receptors or gp120 (HIV surface protein) and ACE2 and then the two kinds of transected cells are mixed at 24 hours. Pictures are taken at 48 hours. The above six pictures indicate that ACE2 is the specific receptor of SARS-CoV, and S318-510 (i.e. the 318^{th} to 510^{th} amino acids of S protein) are the ACE2 binding sites. Other truncated forms of S protein with removed or modified S318-510 section do not react with ACE2 or lead to cell fusion or cause or aggravate acute lung injury, therefore being a safe candidate vaccine.

It is indicated in the invention that preparation of efficient and safe SARS-CoV resistant vaccines demands selection of truncated forms with high performance in initiation of cell-mediated and humoral-mediated immunity reactions from mutated or modified S protein, especially with removal, mutation or modification of the 318^{th} to 510^{th} amino acids of S protein in order to reduce the affinity to ACE receptor or to produce no combination to ACE2 receptor.

The invention relates to the use of said DNA sequence and express amino acid thereof for developing vaccines for preventing SARS-CoV. Said vaccines include DNA vector vaccines, protein vaccines and virus vector vaccines.

### EXAMPLES

### Example 1

### Artificial Synthesis of Full Length Gene of SEQ ID No. 1

The full length of SEQ ID No. 1 is artificially synthesized by the entrusted Shanghai BioAsia Biotech Ltd. (China) with a gene synthesis method known in the art, which adopts oligonucleotide primers of 100 bases and the corresponding PCR amplification primers, wherein the oligonucleotide primers comprises 20 overlapping bases to form a gene, which, after connection, annealing and PCR amplification, is synthesized into a full length gene.

### Example 2

### Construction and Identification of Plasmid

### (1) Acquisition of PCR Products

A. Primer Design and Synthesis

S317:
forword:
5′GGCGCTAGCCAGCGACCTGGACCGCTGC3′
reverse:
5′CGCGGATCCGTCGGGGAAGCGCACGACGTC3′
S510:
forword:
5′GGCGCTAGCCAGCGACCTGGACCGCTGC3′
reverse:
5′CGCGGATCCGTCACGGTGGCGGGGGCGTTC3′
S685:
forword:
5′GGCGCTAGCCAGCGACCTGGACCGCTGC3′
reverse:
5′CGCGGATCCGTGGCGCCCAGGCTCATGGTG3′
S900:
forword:
5′GGCGCTAGCCAGCGACCTGGACCGCTGC3′
reverse:
5′CGCGGATCCGTCTCGTACAGCACGTTCTG3′
S1148:
forword:
5′GGCGCTAGCCAGCGACCTGGACCGCTGC3′
reverse:
5′CGCGGATCCGTCAGGTCCACGTCGGGGCTG3′
S1190:
forword:
5′GGCGCTAGCCAGCGACCTGGACCGCTGC3′
Reverse:
5′CTCACATGTATGGATCCTTCTGCTCGTACTTGCCCAG3′
S318-510:
forword:
5′GGCGCTAGCCATCACCAACCTGTGCCCC3′
reverse:
5′CGCGGATCCGTCACGGTGGCGGGGGCGTTC3′
S318-1190:
forword:
5′GGCGCTAGCCATCACCAACCTGTGCCCC3′
reverse:
5′CTCACATGTATGGATCCTTCTGCTCGTACTTGCCCAG3′
S511-1190:
forword:
5′GGCGCTAGCCTGCGGGCCCAAGCTGAGC3′
reverse:
5′CTCACATGTATGGATCCTTCTGCTCGTACTTGCCCAG3′
S681-1190:
forword:
5′GGCGCTAGCCCTGGGCGCCGACAGCAGC3′
reverse:
5′CTCACATGTATGGATCCTTCTGCTCGTACTTGCCCAG3′

The above primers are all synthesized by Shanghai BioAsia Biotech Ltd. (China).

B. PCR Amplification Products

Amplification reaction is performed on a PCR apparatus (eppendorf Mastercycler, Germany) Primer (1 μg/μl) 0.5 μl

Template PUC18 S 1 μg (after being incubated with restriction endonucleases EcoR1 at 37° C. for an hour)

PCR amplification Kit (2×pfu PCR Master Mix, Cat No: KP-201, Tiangen Biotech (Beijing) Co., Ltd) is used according to the instruction. The materials are added into a 50 μl reaction system and denatured at 94° C. for 5 min. Then 30-40 cycles are repeated as follows: denaturation (94° C., 1 min), annealing (55° C., 30 sec), extension reaction (72° C., 1-2 min). Extension reaction for 10 min at 72° C. is conducted for ending the cycles.

5 μl of the PCR products is analyzed by 1% agarose gel electrophoresis (Agarose, TED&HY Bio Co: Ltd Cat NO: A9918).

The correct products are purified with PCR clean-up kit (VITAGENE, Cat No: 110310-05) and stored in 25 μl TE solution (MOLECULAR CLONING EXPERIMENTAL MANUAL II).

(2) Synthesis of Inserted Fragment

CD5L-top:

CD5L-top:
5′AATTCGCCGCCACCATGCCCATGGGGTCTCTGCAACCGCTGGCCACCT
TGTACCTGCTGGGGATGCTGGTCGCTTCCTGCCTCGGAGCGCTAGCAT
C3′
CD5L-bottom:
5′CATGGATGCTAGCGCTCCGAGGCAGGAAGCGACCAGCATCCCCAGCAG
GTACAAGGTGGCCAGCGGTTGCAGAGACCCCATGGGCATGGTGGCGGCG
3′

The sequences are synthesized by Shanghai BioAsia Biotech Ltd. (China).

Fc fragment sequences are derived from the DNA sequences of human original IgG Fc fragment in GenBank synthesized by Shanghai BioAsia Biotech Ltd.(China).

(3) Construction and Identification of Plasmid

A. The single strand is renatured into double strands as the inserted fragment.

B. Recombinant vector pEAK13 CD5L Fc is constructed.

Vector: pEAK13
Inserted fragment: CD5L, Fc fragment
pEAK13 is used as the vector and digested by restriction endonucleases EcoR I and Not I, then is ligated and transformed into host cells and analyzed (specific procedure is presented as follows) to acquire plasmid pEAK13 CD5L Fc.

C. Recombinant vector pEAK13 CD5L Fc DR is constructed.

Fragment resource: Plasmid containing the gene preserved by our lab
Firstly, the inserted fragment DR is digested from the vector with restriction endonucleases Pst I and Bgl II. Then pEAK13 CD5L Fc is used as the vector for digestion with restriction endonucleases Pst I and Bgl II. Then ligation, transformation and analysis (specific procedure is presented as follow) are conducted to obtain the plasmid pEAK13 CD5L Fc DR.

D. A recombinant vector containing optimized S protein genes and fragments thereof is constructed.

Vector: pEAK13 CD5L Fc DR
Inserted fragments: PCR products S317, S511, S685, S900, S1148, S1190, S318-510, S318-1190, S511-1190, S681-1190.

a. Restriction digestion

The vector DNA or PCR products are added into a restriction endonucleases buffer system and incubated for 1-3 hours. The total volume of the digestion system is 20 μl. Then 1 μg DNA, 2 μl 10×BSA (0.1% BSA), 2 μl 10×NEB Buffer, 0.5 μl restricted endonucleases Nhe I and BamH I (all restricted endonucleases, 0.1% BSA, NEB Buffer are bought from NEW ENGLAND BioLabs® Inc, USA) are added. The digestion of vector DNA needed another 0.5 μl alkali phosphatase (Promega, USA, Cat No: M182A) to remove phosphate from the termini of digested vectors.

8.5 ml low-melting-point gel (Promega, USA, Cat No: v2111) is poured on an electrophoresis glass plate (75×50 mm Pre-Cleaned Micro Slides Plain, corning, USA, No. 2974) with a comb and allowed to congeal.

When the gel is solidified, the comb is removed. And then the gel block is placed in an electrophoresis chamber and added TAE buffer containing 500 ug/L Ethidium Bromide (Promega, USA, Cat: # HS041) (MOLECULAR CLONING EXPERIMENTAL MANUAL II) into. Then 15-20 ul digested vector DNA and PCR product is added into each sample well with the simultaneous addition of DNA marker such as λ-Hind III, DL2000 (TaKaRa Biotechnology (Dalian) Co. Ltd) to determine the sizes of DNA fragments.

Electrophoresis is begun and maintained under 60-80V (electrophoresis apparatus DYY-6C, Beijing Six-One Apparatus Plant) for 20-60 min.

After the electrophoresis, the gel is transferred under the UV (UV analysis apparatus, Beijing New Tech Application Research Institute) and photographed. The needed band is cut off. The cut DNA band is put into 1.5 ml centrifugal tubes and centrifuged for a short time at high speed to sink the gel to the bottom. Then the gel is heated at 65° C. to be melted.

b. Ligation

A ligation buffer system of 40 μl is prepared including 5 μl 10×NEB Buffer 4, 2 μl 100×BSA, 5 μl 10×Ligation Additions, 0.5 μl T4 DNA ligase and 2-4 μl vector DNA in deionized water (T4 DNA ligase, 100×BSA, NEB Buffer are bought from NEW ENGLAND BioLabs® Inc, USA).

The ligation system is divided into two equal portions. 2-4 μl gel the DNA fragment is to be inserted into is added into one portion. Deionized water of the same volume is added into the other portion as the negative control (the ratio of vector to inserted DNA is controlled at 1:2 in the ligation system, and total volume of 1.5% low-melting-point gel didn't exceed 6 μl in each 20 μl system).

The systems are mixed uniform and incubated for 1-3 hours at room temperature.

Transformation competent cell MC1061 is prepared by our lab with the preparation methods of MOLECULAR CLONING EXPERIMENTAL MANUAL II, Preparation of Competent Cells.

c. Transformation

Chemical competent cells are taken out from a −70° C. refrigerator and placed on ice for thawing.

5-6 ml LB agar without ampicillin is poured onto an LB agar culture plate containing 50 μl/ml ampicillin (MOLECULAR CLONING EXPERIMENTAL MANUAL II, Preparation of Solution, Ampicillin (HuaBei Pharmaceutical Factory, China) and allowed to congeal for use.

After the competent cells had just thawed, ligated products and negative control are immediately added in at 5-8 μl every 100 μl competent cells and then mixed softly and positioned on ice for 15-30 min.

Then the system is positioned in water bath of 37° C. for 5 min.

The cell suspension is sucked out and spread uniformly on a culture plate in which LB medium had just been added. After incubation at 37° C. for 12-16 hours, monoclone colonies emerged.

d. Identification

The monoclone colonies are picked with a toothpick and grew in 4 ml LB liquid medium containing ampicillin. The medium is positioned in a 37° C. shaker (Desk-top constant temperature shaker THZ-D, Peiying) and shaked at 250-280 rpm for 7-8 hours until the bacteria suspension became saturated.

Plasmid DNA is extracted with plasmid mini preparation kits (Tiangen Biotech (Beijing) Co., Ltd, DP-103).

The obtained plasmid DNA is dissolved in 50-60 μl TE and digested with 10 μl restriction Nhe I and BamH. After detection, strains in correspondence to plasmids with correct restriction are picked out.

e. Sequencing

The plasmids with correct endonuclease restriction detection are sequenced for further identification (Shanghai BioAsia Biotech Ltd and Shanghai sangon Biological Engineering Technology & Service Co., Ltd).

f. Large-scale plasmid preparation

CsCl density gradient centrifugation is adopted to extract large-scale recombinant plasmids with methods in MOLECULAR CLONING EXPERIMENTAL MANUAL II).

e. Cell transfection and confirmation of high and correct expression of fusion protein

2×10⁵ cells are counted and arranged into each wells of a 6-well culture plate. 24 hours later, the cells are respectively transfected by liposomes (lipofectamine™ 2000 bought from Invitrogen™) with the constructed plasmids containing protein S and various truncated forms thereof. Media at three and six day are collected and detected with Western Blotting procedures for determination of molecular weight of the fusion protein and with flow cytometry technology for determination of the activity of the fusion protein (specific operation methods are presented as follows).

### Example 3

### Construction of Constant Expression Cell Lines

(1) About 10 μg recombinant plasmid is digested by restriction endonucleases AvrII (bought from NEW ENGLAND BioLabs® Inc, USA); a small amount of the digested product is determine whether the digestion is complete by electrophoresis; then the remaining product is purified with purification kits (bought from V-Gene); the DNA is obtained and enzyme and protein removal is carried out.

(2) Cells are digested with trypsin and then blown into single cells with the addition of medium. Then 2×10⁵ cells are counted and arranged into each well of a six-well cell culture plate.

(3) 24 hours later, liposomes (lipofectamine™ 2000 bought from Invitrogen™) are used to transfect water (negative control) and 0.5 μg digested and purified DNA (manipulated according to instruction of kits).

(4) 48 hours later, the cells are distributed into wells of a 12-well culture plate (cells from each well of the six-well plate are arranged into four wells of the 12-well plate); the screening drug puromysin of different concentration (bought from CALBIOCHEM® CLONTECH) is added in gradients to kill cells without DNA transfection.

(5) 72 hours later, cells are selected corresponding to the drug concentration that killed all of the negative control cells and exempted certain cells with DNA transfection. The cells in the wells are treated with limiting dilution assay and single cells are planted in a 96-well cell culture plate.

(6) About 10 days later, certain monoclonal cells are picked out and detected by ELISA and Western Blotting.

By Western Blotting procedure, the molecular weight of the protein is determined for confirmation of the correct expression of genes. The concentration of the protein is determined with ELISA kits for selection of cell lines with high expression. The activity of the protein is determined by flow cytometry technology (specific operation is presented as follows).

### Example 4

### Determination of Molecular Weight of the Fusion Protein by Western Blotting

Equipment and reagents for electrophoresis and membrane transfer:

Electrophoresis apparatus (PowerPac Basic™ Power Supply): bought from BIO-RAD, Catalog Number: 164-5050. Electrophoresis apparatus (Mini-PROTEAN® 3 Cell): bought from BIO-RAD, Catalog Numbers: 165-3301, 165-3302

Electrophoresis transfer apparatus (Mini Trans-Blot® Electrophoretic Transfer Cell): bought from BIO-RAD, Catalog Numbers: 170-3930, 170-3935

Reagents

(SIGMA-ALDRICH CORPORATION·BOX14508·ST. LOUIS·MISSOURI 63178·USA) Specimens are obtained from medium of transfected cells, medium of constructed cell lines and purified protein.

Operation Methods:

Before loading, the samples are added into 2×gel-loading buffer (preparation according to MOLECULAR CLONING EXPERIMENTAL MANUAL II, 890 page, Science Press) with equivalent volume and heated at 97° C. for 5 min.

SDS polyacrylamide gel is prepared with 10% separation gel and 5% concentration gel (preparation according to MOLECULAR CLONING EXPERIMENTAL MANUAL II, 883-884 page, Science Press).

The glass plate is withdrawn from the fixing frame and the gel is mounted in the electrophoresis apparatus according to the instruction; then the whole electrophoresis box is filled full with 1× electrophoresis buffer (MOLECULAR CLONING EXPERIMENTAL MANUAL II, page 884, Science &. Technology Press).

15 μl (7.5 μl supernatant and 7.5 μl loading buffer) samples denatured by pre-heating are carefully injected into gel pores with a micropipette.

According to the instruction, the electrophoresis apparatus is correctly linked and switched on.

The electrophoresis is begun with the initial voltage of 80V. After front end of bromophenol blue dye reached the separation gel, the voltage is increased to 120V until the bromophenol blue dye arrived at the bottom of the separation gel or completely migrated out of the gel. Then the power is switched off. The whole course lasted approximately 120 min.

membrane transferring buffer is prepared (preparation of the solution according to MOLECULAR CLONING EXPERIMENTAL MANUAL II, page 892, Science &. Technology Press) and pre-cooled at 4° C.

When electrophoresis is over, the power is cut off and the electrophoresis box is opened to take the glass plate out. After the concentration gel is cut off, the separation gel is shifted into a container which had been filled with membrane transfer buffer.

A piece of nitrocellulose membrane (Amersham, Catalog No: RPN303C) slightly larger than the separation gel and two pieces of filtering paper of the same size are cut with gloved hands. Then the nitrocellulose membrane, filtering paper and two sponges are respectively soaked into three containers with transfer buffer.

The membrane transfer device is assembled according to instruction of the membrane transfer apparatus. The condition of membrane transfer is conducted under the constant current of 300 mA for 120 min.

After the membrane transfer, the membrane is carefully withdrawn and put into a container with 2% chicken egg albumin sealing liquid (SIGMA® ALBUMIN, CHICKEN EGG, Catalog number: A-5253) and then sealed for one hour at room temperature on a gently shaking shaker (or 4° C., overnight).

After the sealing is completed, the sealing liquid is discarded. The primary antibody is diluted with 2% chicken egg albumin sealing liquid and added for binding for three hours at room temperature or overnight at 4° C.

The membrane is washed with TBST for three times with each time lasting 10 min.

Then the TBST is discarded; and the secondary antibody diluted with 2% chicken egg albumin sealing liquid is added for binding for one to two hours at room temperature (as to Fc etc. which could use secondary antibody directly, the secondary antibody diluted with 2% chicken egg albumin sealing liquid is added after the sealing is completed).

The membrane is washed with TBST for three times with each time lasting 10 min after the antibody binding is over.

The Western detection staining reagent is prepared according to the instruction (Santa Cruz Biotechnology, Inc. Catalog Number: sc-2048) and added uniformly dropwise on one side of membrane having bound with proteins.

The excess staining reagent is absorbed and then the membrane is wrapped and placed into an x-ray photograph dark box (Shantou Yuehua Medical Instrument Co., Ltd, China, Model AX-II, 127×178 mm).

In dark room, the film is exposed in the dark box and developed for 4-5 min, fixed for 4-5 min (film: Kodak X-O mat BT Film, divided and packed by Shantou Kodak Co., Ltd, China, made in America Eastman Kodak Company. 12.7×17.8 cm, emulsion number: 031222104) (developing and fixing powder are bought from Tianjin Hebei Ganguang Cailiao Factory).

The cell monoclone supernatant is qualitatively detected by Western Blotting, bands of the expected size are found.

### Example 5

### Quantitative Detection of Fusion Protein Expression by ELISA

The protein concentration in medium could be detected by ELISA. Reagents of ELISA are from BD Pharmingen™ ELISA kit (BD Biosciences co.)

ELISA Procedures:

(1) Cell supernatant, negative (medium containing bovine serum) and positive controls and standard (gradient diluted human IgG with known concentration, used for quantitative assay of protein) are added into 96-well enzyme-labeled plate with 100 ul for each well and positioned overnight. Each sample is for three wells to discriminate positive results from false positive results.

(2) Next day, the medium is taken out, and the plate is rinsed with wash solution with 200 ul for each well.

(3) Assay diluent is added with 200 ul for each well and shaken on a shaker for one hour at room temperature.

(4) Primary antibody diluted with assay solution is added with 100 ul for each well and shaken on a shaker for 3 hours at room temperature.

(5) After primary antibody binding is over, the plate is rinsed with wash solution for 3 times with 200 ul for each well.

(6) The secondary antibody conjugated with HRP and diluted with assay diluent is added with 100 ul for each well and shaken for one hour at room temperature (as to Fc etc. which could use secondary antibody directly, the secondary antibody diluted with assay diluent is added after the assay diluent binding is completed).

(7) The plate is washed with wash solution for eight times with 200 μl for each well.

(8) Photosensitizers A and B of equal volume are mixed and protected from light and then added to the washed enzyme-labeled plate with 100 ul for each well. Then the plate is protected from light for 30 min at room temperature.

(9) Stop solution is added 30 min later with 50 ul for each well to stop the reaction.

(10) The number is got at 450 nm on an ELISA plate reader.

(11) The concentration of the protein is computerized according to the read number.

The result showed that S protein and truncated forms thereof are expressed more than 10 ug every million cells in 24 hours. Fusion protein S1190-Fc, S1148 Fc, S900 Fc, S685 Fc, S511 Fc, S317 Fc, S318-1190 Fc, S318-510-Fc, S511-1190 Fc, S681-1190Fc are expressed more than 10 μg, 20 μg, 20 μg, 20 μg, 20 μg, 20 μg, 10 μg, 30 μg, 10 μg, 10 μg respectively in the supernatants.

### Example 6

### Assay on Activity of Expression Protein by Flow Cytometry Technology

Vero E6 cell contains ACE2, the acceptor of protein S, which mainly reacts with 318-510 amino acid of S protein. The correctness of protein folding can be confirmed based on the principle that a ligand can bind to corresponding receptor.

Flow Cytometry Procedures:

(1) Vero E6 cells or 293 cells transfected by ACE2 are digested by PBS/2mMEDTA and divided into several portions and placed into centrifuge tubes.

(2) The cells are centrifuged (Eppendorf Centrifuge 5415D) at 1000 rpm for 10 min.

(3) The cells are re-suspended with media containing S protein or the truncated forms respectively; IMDM medium with serum (bought from Hyclone) are used as negative control.

(4) The cells are rotated for mixture for 1-2 hours at 4° C.

(5) The cells are centrifuged (Eppendorf Centrifuge 5415D) at 1000 rpm for 10 min; then the supernatant is discarded.

(6) The cells are re-suspended after addition of secondary antibody FITC/anti-human IgG (bought from Jackson ImmunoResearch) or FITC/anti-His (Sigma) (primary antibody shall be added first if there is no fluorescent labeled secondary antibody).

(7) The cells are rotated for mixture for 30 min to one hour at 4° C.

(8) The cells are centrifuged (BECKMAN COULTER™ Microfuge® 22R Centrifuge) at 1000 rpm for 10 min at 4° C.

(9) The cells are resuspended with PBS and assayed with flow cytometer (BECKMAN COULTER™ EPICS ELITE EST).

### Example 7

### Assay of Down Regulation of ACE2 Acceptor by Flow Cytometry

Vero E6 cell contains ACE2, the acceptor of S protein. So Vero E6 cells can be used to detect down regulation effect of S protein on ACE2.

Procedures:

(1) The medium (containing 10% FB (Hyclone)) of the 10 cm petri dish (Greiner bio-one) with 50-70% filled with Vero E6 cells is removed. Then the petri dish is rinsed with PBS for three times.

(2) Serum free medium is added and incubated in a 37° C. CO2 incubator (SANYO, MCO-15AC) for 1 h.

(3) The petri dish is rinsed with PBS once and added 2 mM EDTA/PBS in, and then incubated in a 37° C. CO₂ incubator for 20-30 min.

(4) The cells rounding up are blown off and divided into 3 portions.

(5) The cells are centrifuged at 1000 rpm for 10 min (BECKMAN COULTER™, Microfuge® 22R Centrifuge) and then resuspended with 800 μl serum free medium with appropriate EDTA addition.

(6) Control Fc is added into one portion and 50 ug fusion protein S1190-Fc is respectively added into the other two.

(7) The portion with control Fc addition and one portion with S1190-Fc are rotated slowly at 4° C. and the other portion with S1190-Fc is rotated at 37° C. The step lasted 3 hours.

(8) The three portions are centrifuged at 1000 rmp for 10 min at 4° C.

(9) After re-suspension with PBS, the portions are centrifuged at 1000 rmp for 10 min at 4° C.

(10) The FITC labeled anti-Fc antibodies are diluted in PBS and then the cells are re-suspended (when ACE2 detected, primary antibody of ACE2 should be added first, then FITC labeled secondary antibody).

(11) The cells are rotated gently for 30 min at 4° C.

(12) The cells are centrifuged at1000 rmp for 10 min at 4° C.

After resuspension in PBS, the cells are detected by flow cytometer (BECKMAN COULTER™ EPICS ELITE EST).

### Example 8

### Cell Fusion Experiment

(1) 293ET cells at log phase are digested by trypsin. After the cells rounded up, DMEM medium (bought from GIBCO) is added to blow and scatter the cells.

(2) 2×105 cells are counted and distributed into each well of a 6-well plate.

(3) 24 hours later, the plasmid is respectively transfected with liposomes (lipofectamine™ 2000 bought from Invitrogen™).

(4) 24 hours later, the cells are digested by trypsin and counted; every two cells are mixed and placed into each well of a 12-well plate. The amount of each type of cells per well is 2×104, 4×104, 6×104, 8×104, 1×105.

(5) Significant cell fusion is observed in the photograph taken 48-72 hours later (Nikon Eclipse TE2000-U); however, no fusion is observed in the negative control.

### Example 9

### Detection of Protein S1190 and ACE2 Interaction with IP Test

(1) Cells are transfected with two plasmid units including S1190-Fc and ACE2 as well as Fc and ACE2 with the latter as the control.

(2) After 36 hours' transfection, the cells are placed on ice for pre-cooling and washed with precooled PBS for 3 times.

(3) Cell lysis solution containing protease inhibitor is added and the lysis is allowed to last for 20-30 min.

(4) The cells and the lysis solution are collected and centrifuged (BECKMAN COULTER™, Microfuge® 22R Centrifuge) at 12000 rpm for 2 min at 4° C.

(5) The supernatant is transferred to a new tube; then adequate Protein G-Agarose is added and rotated slowly at 4° C. overnight.

(6) The supernatant with magnetic beads are centrifuged at 12000 rpm for 5 min at 4° C.

(7) After the supernatant is discarded, the cells are resuspended in adequate lyses solution and rotated slowly for 20 min.

(8) The cells are centrifuged at 12000 rpm for 5 min at 4° C.

(9) The supernatant is discarded and then 2× Western Blotting loading buffer of same volume with sediment is added and stored at 97° C. for 5 min.

(10) The system is centrifuged at high speed; the supernatant is taken for detection by Western Blotting.

### Example 10

### Protein Purification

The Fc labeled protein is purified with a protein A columns, and 6His tag protein is purified with a nickel column.

The Fc labeled protein with a protein A column produced by Amersham (Biosciences AB, Sweden; CAT NO: 17-04020-03).

(1) Supernatant of constant expression cell lines which had been cultured for three days are collected.

(2) Dialysis: The collected supernatant is dialyzed. The dialysis solution contained 11.54 mM/L Na2HPO4, 8.46 mM/L NaH2PO4 (Beijing chemistry Factory, China) and 1 mM EDTA (Promega U.S.A) and has the pH of 7.0. The dialysis lasted at least 8 hours and volume of dialysis solution is no less than 20 times that of the supernatant.

(3) Filtration: The dialyzed liquid is filtered with 0.45 μm Durapore membrane filters (Millipore, Ireland; CAT NO: HVLP04700).

(4) Purification: The purification is carried out according to the protocol in product instruction of Amersham with Econo Gradient Pump Kits (Bio-Rad U.S.A).

(5) The purified protein sample is analyzed by Western Blotting and Coomassie brilliant blue staining of SDS-polyacrylamide gel. Concentration of protein is determined by Lowry (Lowry kits are bought from Tianxiang Bangding Co Ltd, CAT NO: TB090-1).

Purification of 6His tag protein is carried out in the same way.

### Example 11

### Detection of Neutralizing Antibody of Vaccine in Serum

Titers of neutralizing antibody are produced in mice after immunized by S1190-Fc.

(1) Five weeks old female balb/c mice are divided into two groups with each containing five.

(2) One group is injected with 50 ug S1190-Fc with equivalent Freud's adjuvants at 0, 2, 4 weeks respectively, the other is administrated with Fc and equivalent Freud's adjuvants as the control.

(3) Serum is collected at 2, 4, 6 weeks.

(4) The serum after thermal inactivation is doubly diluted.

(5) The titers of neutralizing antibody are detected and analyzed by micro-amount neutralization assay.

The neutralizing antibody is added in gradient into a 96-well plate with each gradient for three wells. Then SARS-CoV is added at the dosage of 100 times the TCID50 of infecting monolayer adherent cell Vero E6. The cytopathic effect (CPE) is detected at the third and the fourth days. The concentration at which CPE could be inhibited thoroughly in 50% wells is calculated with the RM formula. Finally the titer of neutralizing antibody is obtained.

### Example 12

### Lung Elastance Test

(1) 2.5-3 month old mice are divided into 5 groups with each containing 5-7 mice.

(2) The mice are anesthetized by intraperitoneal injection of ketamin (75 mg/kg) and xylazine (20 mg/kg).

(3) After tracheotomy, the ventilatory capacity is measured with a flow-stable ventilator with controllable air current.

(4) The record of air current is normalized with VRM and considered as baseline of measurement.

(5) The mice are intraperitoneally injected with S1190-Fc, S318-510-Fc, or control Fc (5.5 nmol/kg) respectively 30 min before acid or saline solution treatment.

(6) The mice are conducted intratrachea inoculation with hydrochloric acid or saline solution; then VRM (35 cmH2O, 3 seconds) is determined. All the animals are ventilated for 3 hours (FIO2 1.0) and the analysis of lung elastance is recorded.

(7) The total PEEP (PEEPt) is measured at end expiration and inhalation obstruction after the pressure became stable (Pplat) as (Pplat minus PEEPt)/VT; the lung elastance is calculated every 30 min during the ventilation.

8) After 1-2 hour acid or saline solution treatment, the mice are again intraperitoneally injected with S1190-Fc, S318-510-Fc, or control Fc (5.5 nmol/kg) respectively.

### Example 13

### Immunohistochemistry Assay of Mice

(1) Right lungs of the mice in Example 12 are taken as the specimen. The lung tissue is fixed with 3.7% formaldehyde and embedded with paraffin.

(2) The lung tissue is cut into 5 μm sections.

(3) The tissue sections are pretreated with 72° C. EDTA.

The tissue sections are stained with goat anti-human polyclonal antibody (Jackson Immunological Research, Inc.) and the specific stained parts are detected with Vectastatin ABC kits.

### Example 14

### H&E Staining

(1) Acid or saline solution treatment is carried out as in Example 12 and the mice are intraperitoneally injected with S1190-Fc or control Fc.

(2) Right lungs of the mice in Example 12 are taken as the specimen. The lung tissue is fixed with 3.7% formaldehyde and embedded with paraffin.

(3) The lung tissue is cut into 5 μm thick sections.

(4) The tissue sections are stained with haematoxylin and eosin.

(5) The tissue sections are photographed under a microscope.

### Example 15

### Lung Damage Scoring

Semi-quantitative measurement of lung damage of the mice treated by S1190-Fc and control Fc after acid inhalation is carried out.

(1) Four visual fields are randomly selected from each section in Example 14. 16 fields in each group are scored blindly according to the scoring standard.

(2) Content of scoring included pulmonary alveolus hyperemia, hemorrhage, neutrophilic leukocyte infiltration, thickness of alveolar wall, and pulmonary hyaline membrane formation etc.

(3) Scoring standard: minimal damage: 0, slight damage: 1, mild damage: 2, severe damage: 3, maximal damage: 4.

1. A fusion protein of structural protein of SARS-CoV virus having a formula of X-Y-Z, wherein,
X comprises the structural protein S, M, E or N of SARS-CoV virus, or any shorten forms of the said structural proteins, the structural protein S comprises fragments in which any fragment of the amino acids 318 to 510 is removed, modified or mutated, or fragment in which the amino acids 318 to 510 is removed, modified or mutated;
Y is a linking part consisting of 0-20 any amino acids;
Z is a Fc, its variants of human IgG₁ including hinge region, CH₂, CH₃ region or protein tags.

2. The fusion protein of structural protein of SARS-CoV virus according to claim 1, characterized in that the said protein tags comprises the 6×His tag, the PEG tag and the Human serum albumin (HAS) tag.

3. The fusion protein of structural protein of SARS-CoV virus according to claim 1, characterized in that the said structural protein S of SARS-CoV virus comprises the full-length protein S and any shorten forms thereof.

4. The fusion protein of structural protein of SARS-CoV virus according to claim 1, characterized in that the said structural protein S of SARS-CoV virus is unable to bind to its receptor ACE2 or weaken the binding ability to its receptor ACE2.

5. The fusion protein of structural protein of SARS-CoV virus according to claim 1, characterized in that the said Y has 2 amino acids, and the amino acids are lysine and arginine.

6. A gene encoding the structural protein S of SARS-CoV virus capable of being expressed in mammal cell lines, characterized in that its nucleotide sequence is shown as SEQ ID NO: 1.

7. A recombinant expression plasmids comprising the gene of claim 6 having the sequence of SEQ ID NO: 1.

8. The recombinant expression plasmids according to claim 7, characterized in that the said plasmids comprises Eukaryotic PEAK series.

9. A Mammalian cell lines comprising a gene encoding the structural protein S of SARS-CoV virus capable of being expressed in mammal cell lines, characterized in that its nucleotide sequence is shown as SEQ ID NO: 1, and being capable of expressing the fusion protein as claimed in claim 1.

10. The Mammalian cell lines according to claim 9, characterized in that the said cell lines comprise CHO, 293 and Vero cell lines and derived cell lines thereof.

11. The Mammalian cell lines according to claim 10, characterized in that the said cell lines are deposited at China General Microbiological Culture Collection Center (CGMCC), the deposited Nos. are respectively 1408, 1409 and 1410.

12. A method for producing the fusion protein of structural protein of SARS-CoV virus as claimed in claim 1, comprising the steps of:
(1) transfecting a recombined expression plasmid which expresses a fusion proteins as claimed in claim 1 and endogenous dihydrofolate reductase (dhfr) and constructing mammalian expression cell lines;
(2) producing over 10 μg of recombined proteins per million cells in the mammalian expression cell lines under normal growth circumstances in 24 hours; and
(3) Purifying the recombined proteins expressed in step (2).

13. The method according to claim 12, characterized in that the said recombined expression plasmid comprises a leader sequence that is a leader sequence of protein CD5.

14. The method according to claim 12, characterized in that the said recombined expression plasmid has genes encoding the structural protein of SARS-CoV virus, the said genes are artificial synthesized by using the common or use bias codons for human cells to replace the use bias codons for virus which encode the same amino acids, optimize the codons of the virus structural proteins to use the human use bias codons.

15. The method according to claim 12, characterized in that the gene encoding the structural protein S of SARS-CoV virus is optimized by using the common or use bias codons for human cells, and the sequence for the said gene is shown as SEQ ID NO:1.

16. The method according to claim 12, characterized in that the said mammalian expression cell lines comprises CHO, 293 and Vero cell lines and derived cell lines thereof.

17. The method according to claim 16, characterized in that the said mammalian expression cell lines are deposited at China General Microbiological Culture Collection Center (CGMCC), and the deposited Nos. are respectively 1408, 1409 and 1410.

18. The method according to claim 12, characterized in that the screening drugs used in constructing mammalian expression cell lines comprise puromycin and amethopterin.

19. The method according to claim 12, characterized in that in the step (2), 30 μg or more recombined proteins are produced in medium by each million cells of the mammalian expression cell lines under normal growth circumstances in 24 hours.

20. The method according to claim 12, further comprising using the fusion protein in at least one of the following:
manufacturing a vaccine for prophylaxis of SARS-CoV virus infection;
manufacturing a kit for detecting SARS-CoV virus infection;
manufacturing a medicament for preventing, inhibiting, or treating SARS-CoV virus infection
screening a medicament for preventing, inhibiting, or treating SARS-CoV virus infection; or
manufacturing an antibody for preventing SARS-CoV virus infection.

21.-27. (canceled)


SEQUENCE LISTING (24 sequences)

SEQ ID NO: 1 - DNA, 3740 bp, artificial sequence
  misc_feature (1)..(3740): chemically synthesized
ctagccagcgacctggaccgctgcaccaccttcgacgacgtgcaggcccccaactacacccagcacaccagcagcatgcgcggcgtgtactaccccgacgagattttccgcagcgacaccctgtacctgacccaggacctgttcctgcccttctacagcaacgtgaccggcttccacaccatcaaccacaccttcggcaaccccgtgatccccttcaaggacggcatctacttcgccgccaccgagaagagcaacgtggtccgcggctgggtgttcggcagcaccatgaacaacaagtcccagtccgtgatcatcatcaacaacagcaccaacgtggtgatccgcgcctgcaacttcgagctgtgcgacaaccccttcttcgccgtgagcaagcctatggggacccagacccacaccatgatcttcgacaacgccttcaactgcaccttcgagtacatcagcgacgccttcagcctggacgtgagcgagaagagcggcaacttcaagcacctgcgcgagttcgtgttcaagaacaaggacggcttcctgtacgtgtacaagggctaccagcccatcgacgtggtgcgcgacctgcccagcggcttcaacaccctgaagcccatcttcaagctgcccctgggcatcaacatcaccaacttccgcgccatcctgaccgccttcagccccgcccaggacatctggggcacctccgccgccgcctacttcgtgggctacctgaagcccaccaccttcatgctgaagtacgacgagaacggcaccatcaccgatgccgtcgactgcagccagaaccccctggccgagctgaagtgcagcgtgaagagcttcgagatcgacaagggcatctaccagaccagcaacttccgcgtggtgcccagcggcgacgtcgtgcgcttccccaacatcaccaacctgtgccccttcggcgaggtgttcaacgccaccaagttccccagcgtgtacgcctgggagcgcaagaagatctccaactgcgtggccgactacagcgtgctgtacaacagcaccttcttcagcaccttcaagtgctacggcgtgagcgccaccaagctgaacgacctgtgcttcagcaacgtgtacgccgacagcttcgtcgtgaagggcgacgacgtgcgccagatcgcccccggccagaccggcgtgatcgccgactacaactacaagctgcccgacgacttcatgggctgcgtgctggcctggaacacccgcaacatcgacgccaccagcaccggcaactacaactacaagtaccgctacctgcgccacggcaagctgcgccccttcgagcgcgacatcagcaacgtgcccttcagccccgacggcaagccctgcaccccccccgccctgaactgctactggcccctgaacgactacggcttctacaccaccaccggcatcggctaccagccctaccgcgtggtggtgctgagcttcgagctgctgaacgcccccgccaccgtgtgcgggcccaagctgagcaccgacctgatcaagaaccagtgcgtgaacttcaacttcaacggcctgaccggcaccggcgtcctgacccccagcagcaagcgcttccagcccttccagcagttcgggcgcgacgtgagcgacttcaccgacagcgtgcgcgaccccaagaccagcgagatcctggacatcagcccctgcgccttcggcggcgtgagcgtgatcacccccggcaccaacgccagcagcgaggtggccgtgctgtaccaggacgtgaactgcaccgacgtgagcaccgccatccacgccgaccagctgacccccgcctggcgcatctacagcaccggcaacaacgtgttccagacccaggccgggtgcctgatcggcgccgagcacgtggacaccagctacgagtgcgacatccccatcggggccgggatctgcgccagctaccacaccgtgagcctgctgcgcagcaccagccagaagagcatcgtggcctacaccatgagcctgggcgccgacagcagcatcgcctacagcaacaacaccatcgccatccccaccaacttcagcatcagcatcaccaccgaggtgatgcccgtgagcatggccaagaccagcgtggactgcaatatgtacatctgcggcgacagcaccgagtgcgccaacctgctgctgcagtacggcagcttctgcacccagctcaaccgcgccctgagcggcatcgccgccgagcaggaccgcaacacccgcgaggtgttcgcccaggtgaagcagatgtacaagacccccaccctgaagtacttcggcggcttcaacttcagccagatcctgcccgaccccctgaagcccaccaagcgcagcttcatcgaggacctgctgttcaacaaggtgactctggccgacgccggcttcatgaagcagtacggcgagtgcctgggcgacatcaacgcccgcgacctgatctgcgcccagaagttcaacggcctgaccgtgctgccccccctgctgaccgacgacatgatcgccgcctacaccgccgccctggtgagcggtaccgccaccgccggctggaccttcggcgccggcgccgccctgcagatccccttcgccatgcagatggcctaccgcttcaacggcatcggggtgacccagaacgtgctgtacgagaaccagaagcagatcgccaaccagttcaacaaggccatcagccagatccaggagagcctgaccaccaccagcaccgccctgggcaagctgcaggacgtggtcaaccagaacgcccaggccctgaacaccctggtgaagcagctcagcagcaacttcggcgccatcagcagcgtgctgaacgacatcctgagccgcctggacaaggtggaggccgaggtgcagatcgaccgcctgatcaccggccgcctgcagagcctgcagacctacgtgacccagcagctcatccgcgccgccgagatccgcgccagcgccaacctggccgccaccaagatgagcgagtgcgtgctgggccagagcaagcgcgtggacttctgcggcaagggctaccacctgatgagcttcccccaggccgccccccacggcgtggtgttcctgcacgtcacctacgtgcccagccaggagcgcaacttcaccaccgcccccgccatctgccacgagggcaaggcctacttcccccgcgagggcgtgttcgtgttcaacgggaccagctggttcatcacccagcgcaacttcttcagcccccagatcatcaccaccgacaacaccttcgtgagcggcaactgcgacgtggtgatcggcatcatcaacaacaccgtgtacgaccccctgcagcccgagctggacagcttcaaggaggagctggacaaatacttcaagaaccacaccagccccgacgtggacctgggcgacatcagcggcatcaacgccagcgtggtgaacatccagaaggagatcgaccgcctgaacgaggtcgccaagaacctgaacgagagcctgatcgacctgcaggagctgggcaagtacgagcagtacatcaagtggccctggtacgtgtggctgggcttcatcgccggcctgatcgccatcgtgatggtgactatcctgctgtgctgcatgacctcctgctgctcctgcctgaagggcgcctgctcctgcggctcctgctgcaagttcgacgaggacgacagcgagcccgtgctgaagggcgtgaagctgcactacaccaaggatcc

SEQ ID NO: 2 - DNA, 75 bp, artificial sequence
  misc_feature (1)..(75): chemically synthesized
atgcccatggggtctctgcaaccgctggccaccttgtacctgctggggatgctggtcgcttcctgcctcggagcg

SEQ ID NO: 3 - DNA, 28 bp, artificial sequence
  misc_feature (1)..(28): chemically synthesized
ggcgctagccagcgacctggaccgctgc

SEQ ID NO: 4 - DNA, 30 bp, artificial sequence
  misc_feature (1)..(30): chemically synthesized
cgcggatccgtcggggaagcgcacgacgtc

SEQ ID NO: 5 - DNA, 28 bp, artificial sequence
  misc_feature (1)..(28): chemically synthesized
ggcgctagccagcgacctggaccgctgc

SEQ ID NO: 6 - DNA, 30 bp, artificial sequence
  misc_feature (1)..(30): chemically synthesized
cgcggatccgtcacggtggcgggggcgttc

SEQ ID NO: 7 - DNA, 28 bp, artificial sequence
  misc_feature (1)..(28): chemically synthesized
ggcgctagccagcgacctggaccgctgc

SEQ ID NO: 8 - DNA, 30 bp, artificial sequence
  misc_feature (1)..(30): chemically synthesized
cgcggatccgtggcgcccaggctcatggtg

SEQ ID NO: 9 - DNA, 28 bp, artificial sequence
  misc_feature (1)..(28): chemically synthesized
ggcgctagccagcgacctggaccgctgc

SEQ ID NO: 10 - DNA, 29 bp, artificial sequence
  misc_feature (1)..(29): chemically synthesized
cgcggatccgtctcgtacagcacgttctg

SEQ ID NO: 11 - DNA, 28 bp, artificial sequence
  misc_feature (1)..(28): chemically synthesized
ggcgctagccagcgacctggaccgctgc

SEQ ID NO: 12 - DNA, 30 bp, artificial sequence
  misc_feature (1)..(30): chemically synthesized
cgcggatccgtcaggtccacgtcggggctg

SEQ ID NO: 13 - DNA, 28 bp, artificial sequence
  misc_feature (1)..(28): chemically synthesized
ggcgctagccagcgacctggaccgctgc

SEQ ID NO: 14 - DNA, 37 bp, artificial sequence
  misc_feature (1)..(37): chemically synthesized
ctcacatgtatggatccttctgctcgtacttgcccag

SEQ ID NO: 15 - DNA, 28 bp, artificial sequence
  misc_feature (1)..(28): chemically synthesized
ggcgctagccatcaccaacctgtgcccc

SEQ ID NO: 16 - DNA, 30 bp, artificial sequence; identical to SEQ ID NO: 6
  misc_feature (1)..(30): chemically synthesized

SEQ ID NO: 17 - DNA, 28 bp, artificial sequence
  misc_feature (1)..(28): chemically synthesized
ggcgctagccatcaccaacctgtgcccc

SEQ ID NO: 18 - DNA, 37 bp, artificial sequence; identical to SEQ ID NO: 14
  misc_feature (1)..(37): chemically synthesized

SEQ ID NO: 19 - DNA, 28 bp, artificial sequence
  misc_feature (1)..(28): chemically synthesized
ggcgctagcctgcgggcccaagctgagc

SEQ ID NO: 20 - DNA, 37 bp, artificial sequence; identical to SEQ ID NO: 14
  misc_feature (1)..(37): chemically synthesized

SEQ ID NO: 21 - DNA, 28 bp, artificial sequence
  misc_feature (1)..(28): chemically synthesized
ggcgctagccctgggcgccgacagcagc

SEQ ID NO: 22 - DNA, 37 bp, artificial sequence; identical to SEQ ID NO: 14
  misc_feature (1)..(37): chemically synthesized

SEQ ID NO: 23 - DNA, 97 bp, artificial sequence
  misc_feature (1)..(97): chemically synthesized
aattcgccgccaccatgcccatggggtctctgcaaccgctggccaccttgtacctgctggggatgctggtcgcttcctgcctcggagcgctagcatc

SEQ ID NO: 24 - DNA, 97 bp, artificial sequence
  misc_feature (1)..(97): chemically synthesized
catggatgctagcgctccgaggcaggaagcgaccagcatccccagcaggtacaaggtggccagcggttgcagagaccccatgggcatggtggcggcg